(19) United States
(12) Patent Application Publication
(10) Pub. No.: US 2009/0143396 A1
(43) Pub. Date: Jun. 4, 2009

(54) Sulfonyl-Substituted Aryl Compounds as Modulators of Peroxisome Proliferator Activated Receptors

(76) Inventors: James W. Malecha, San Diego, CA (US); Andrew K. Lindstrom, Encinitas, CA (US); Sergio G. Duron, San Diego, CA (US); David J. Thomas, San Diego, CA (US)

Correspondence Address: GLOBAL PATENT GROUP - KAL;ATTN: MS LAVERN HALL, 10411 Clayton Road, Suite 304, St. Louis, MO, 63131, US

(21) Appl. No.: 12/083,430

(22) PCT Filed: Oct. 11, 2006
(86) PCT No.: PCT/US2006/040075

§ 371 Date: Apr. 24, 2008

Related U.S. Application Data
(60) Provisional application No. 60/819,510, filed on Jul. 7, 2006.
(60) Provisional application No. 60/726,401, filed on Oct. 12, 2005.

(51) Int. Cl. A61K 31/495; C07D 295/26; A61P 3/10

(52) U.S. Cl. 514/252.12; 544/383

(57) ABSTRACT

Compounds as modulators of peroxisome proliferator activated receptors, pharmaceutical compositions comprising the same, and methods of treating disease using the same are disclosed.

### CROSS REFERENCE TO RELATED APPLICATION

This application claims the benefit of priority of U.S. provisional applications Ser. Nos. 60/726,401, filed on Oct. 12, 2005 and 60/819,510, filed Jul. 7, 2006, the disclosures of which are hereby incorporated by reference as if written herein in their entirety.

### FIELD OF THE INVENTION

The present invention relates to novel sulfonyl-substituted bicyclic aryl derivatives and methods for treating various diseases by modulation of nuclear receptor mediated processes using these compounds, and in particular processes mediated by peroxisome proliferator activated receptors (PPARs).

### BACKGROUND OF THE INVENTION

Peroxisome proliferators are a structurally diverse group of compounds which, when administered to mammals, elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, Ann. Rev. Cell Biol. 1:489-530 (1985); Vamecq and Draye, Essays Biochem. 24:1115-225 (1989); and Nelali et al., Cancer Res. 48:5316-5324 (1988)). Compounds that activate or otherwise interact with one or more of the PPARs have been implicated in the regulation of triglyceride and cholesterol levels in animal models. Compounds included in this group are the fibrate class of hypolipidemic drugs, herbicides, and phthalate plasticizers (Reddy and Lalwani, Crit. Rev. Toxicol. 12:1-58 (1983)). Peroxisome proliferation can also be elicited by dietary or physiological factors such as a high-fat diet and cold acclimatization.

Biological processes modulated by PPAR are those modulated by receptors, or receptor combinations, which are responsive to the PPAR receptor ligands. These processes include, for example, plasma lipid transport and fatty acid catabolism, regulation of insulin sensitivity and blood glucose levels, which are involved in hypoglycemia/hyperinsulinemia (resulting from, for example, abnormal pancreatic beta cell function, insulin secreting tumors and/or autoimmune hypoglycemia due to autoantibodies to insulin, the insulin receptor, or autoantibodies that are stimulatory to pancreatic beta cells), macrophage differentiation which lead to the formation of atherosclerotic plaques, inflammatory response, carcinogenesis, hyperplasia, and adipocyte differentiation.

Subtypes of PPAR include PPAR-alpha, PPAR-delta (also known as NUC1, PPAR-beta and FAAR) and two isoforms of PPAR-gamma. These PPARs can regulate expression of target genes by binding to DNA sequence elements, termed PPAR response elements (PPRE). To date, PPRE's have been identified in the enhancers of a number of genes encoding proteins that regulate lipid metabolism suggesting that PPARs play a pivotal role in the adipogenic signaling cascade and lipid homeostasis (H. Keller and W. Wahli, Trends Endoodn. Met. 291-296, 4 (1993)).

Insight into the mechanism whereby peroxisome proliferators exert their pleiotropic effects was provided by the identification of a member of the nuclear hormone receptor superfamily activated by these chemicals (Isseman and Green, Nature 347-645-650 (1990)). The receptor, termed PPAR-alpha (or alternatively, PPARα), was subsequently shown to be activated by a variety of medium and long-chain fatty acids and to stimulate expression of the genes encoding rat acyl-CoA oxidase and hydratase-dehydrogenase (enzymes required for peroxisomal β-oxidation), as well as rabbit cytochrome P450 4A6, a fatty acid ω-hydroxylase (Gottlicher et al., Proc. Natl. Acad. Sci. USA 89:4653-4657 (1992); Tugwood et al., EMBO J 11:433-439 (1992); Bardot et al., Biochem. Biophys. Res. Comm. 192:37-45 (1993); Muerhoff et al., J Biol. Chem. 267:19051-19053 (1992); and Marcus et al., Proc. Natl. Acad Sci. USA 90(12):5723-5727 (1993).

Activators of the nuclear receptor PPAR-gamma (or alternatively, PPARγ), for example troglitazone, have been clinically shown to enhance insulin-action, to reduce serum glucose and to have small but significant effects on reducing serum triglyceride levels in patients with Type 2 diabetes. See, for example, D. E. Kelly et al., Curr. Opin. Endocrinol. Diabetes, 90-96, 5 (2), (1998); M. D. Johnson et al., Ann. Pharmacother., 337-348, 32 (3), (1997); and M. Leutenegger et al., Curr. Ther. Res., 403-416, 58 (7), (1997).

PPAR-delta (or alternatively, PPARδ) initially received much less attention than the other PPARs because of its ubiquitous expression and the unavailability of selective ligands. However, genetic studies and recently developed synthetic PPAR-δ agonists have helped reveal its role as a powerful regulator of fatty acid catabolism and energy homeostasis. Studies in adipose tissue and muscle have begun to uncover the metabolic functions of PPAR-δ. Transgenic expression of an activated form of PPAR-δ in adipose tissue produces lean mice that are resistant to obesity, hyperlipidemia and tissue steatosis induced genetically or by a high-fat diet. The activated receptor induces genes required for fatty acid catabolism and adaptive thermogenesis. Interestingly, the transcription of PPAR-γ target genes for lipid storage and lipogenesis remain unchanged. In parallel, PPAR-δ-deficient mice challenged with a high-fat diet show reduced energy uncoupling and are prone to obesity. Together, these data identify PPAR-δ as a key regulator of fat-burning, a role that opposes the fat-storing function of PPAR-γ. Thus, despite their close evolutionary and structural kinship, PPAR-γ and PPAR-δ regulate distinct genetic networks. In skeletal muscle, PPAR-δ likewise upregulates fatty oxidation and energy expenditure, to a far greater extent than does the lesser-expressed PPAR-α. (Evans R M et al 2004 Nature Med 1-7, 10 (4), 2004)

PPAR-δ is broadly expressed in the body and has been shown to be a valuable molecular target for treatment of dyslipidemia and other diseases. For example, in a recent study in insulin-resistant obese rhesus monkeys, a potent and selective PPAR-delta compound was shown to decrease VLDL and increase HDL in a dose response manner (Oliver et al., Proc. Natl. Acad. Sci. U.S.A. 98: 5305, 2001).

Because there are three isoforms of PPAR and all of them have been shown to play important roles in energy homeostasis and other important biological processes in human body and have been shown to be important molecular targets for treatment of metabolic and other diseases (see Willson, et al. J. Med. Chem. 43: 527-550 (2000)), it is desired in the art to identify compounds which are capable of interacting with multiple PPAR isoforms or compounds which are capable of selectively interacting with only one of the PPAR isoforms, preferably PPARδ. Such compounds would find a wide variety of uses, such as, for example, in the treatment or prevention of obesity, for the treatment or prevention of diabetes, dyslipidemia, metabolic syndrome X and other uses.

Several PPAR-modulating drugs have been approved for use in humans. Fenofibrate and gemfibrozil are PPARα modulators; pioglitazone (Actos, Takeda Pharmaceuticals and Eli Lilly) and rosiglitazone (Avandia, GlaxcoSmithKline) are PPARγ modulators. However, all of these compounds have liabilities as potential carcinogens, having been demonstrated to have proliferative effects leading to cancers of various types (colon; bladder with PPARα modulators and liver with PPARγ modulators) in rodent studies. Therefore, a need exists to identify other modulators of PPARs which lack these liabilities. Selective modulators of PPARδ may provide an opportunity for such improvements, and may even prove useful in the treatment of cancers, including colon, skin, and lung cancers.

### SUMMARY OF THE INVENTION

The present invention relates to sulfonyl-substituted bicyclic compounds, useful as modulators of PPAR and methods of treating metabolic disorders. One embodiment of the invention are compounds having the structure of Formula (I)

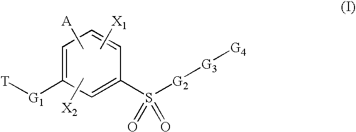

or a salt, ester, or prodrug thereof, wherein:

T is —C(O)OH, —C(O)NH₂, or tetrazole;

G₁ is selected from the group consisting of —(CR₁R₂)ₙ—, -Z(CR₁R₂)ₙ—, —(CR₁R₂)ₙZ, and —(CR₁R₂)ᵣZ(CR₁R₂)ₛ—;

Z is O, S, or NR₃;

n is to 5;

r and s are each independently 0 or 1;

R₁ and R₂ are each independently selected from the group consisting of hydrogen, halogen, optionally substituted lower alkyl, optionally substituted lower heteroalkyl, and optionally substituted lower alkoxy; or alternatively, R₁ and R₂ together may form an optionally substituted cycloalkyl;

R₃ is selected from the group consisting of hydrogen, optionally substituted lower alkyl, and optionally substituted heteroalkyl;

A, X₁, and X₂ are each independently selected from the group consisting of hydrogen, halogen, hydroxy, optionally substituted lower alkyl, optionally substituted cycloalkyl, optionally substituted heteroalkyl, optionally substituted heterocycloalkyl, optionally substituted lower alkynyl, perhaloalkyl, perhaloalkoxy, optionally substituted lower alkoxy, nitro, cyano, and optionally substituted amino;

G₂ is a 5, 6, or 7-membered carbocycle or heterocycle having the structure

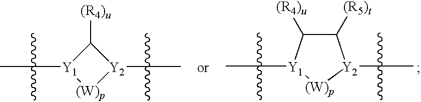

Y₁ and Y₂ are each independently selected from the group consisting of CR₆ and N;

R₄ and R₅ are each independently selected from the group consisting of hydrogen, halogen, hydroxy, optionally substituted lower alkyl, lower perhaloalkyl, optionally substituted heteroalkyl, optionally substituted cycloalkyl, optionally substituted lower alkoxy, nitro, cyano, lower perhaloalkoxy, NH₂, and —C(O)—O—R₁₁; or, when both Y₁ and Y₂ are N, one of R₄ or R₅ may be taken together with one of W to form an optionally substituted 1- or 2-carbon bridge;

R₁₁ is selected from the group consisting of hydrogen and optionally substituted lower alkyl;

W is selected from the group consisting of —CR₇R₈—, and —CR₇— joined together with Y₁ or Y₂ by a double bond;

R₆ is selected from the group consisting of hydrogen, optionally substituted lower alkyl, hydroxy, and lower perhaloalkyl, or is null when Y₁ or Y₂ is joined to W by a double bond;

u and t are each independently 1 or 2;

R₇ and R₈ are each independently selected from the group consisting of hydrogen, optionally substituted lower alkyl, optionally substituted cycloalkyl, optionally substituted heteroalkyl, hydroxy, optionally substituted lower alkoxy, cyano, halogen, lower perhaloalkyl, NH₂, and a moiety which taken together with R₄ and R₅ forms a 1 or 2 carbon bridge;

p is 1, 2 or 3;

G₃ is selected from the group consisting of —(CR₉R₁₀)ₘ—, and —(CR₉R₁₀)_{q}J(CR₉R₁₀)ₖ—,

J is O, S, SO₂, C(O) or NR₁₂;

m is 1 to 3;

q is 0 to 3;

k is 0 to 3;

R₉ and R₁₀ are each independently selected from the group consisting of hydrogen, optionally substituted lower alkyl, optionally substituted lower alkoxy, lower perhaloalkyl, cyano, and nitro;

R₁₂ is selected from the group consisting of hydrogen, optionally substituted lower alkyl, and optionally substituted heteroalkyl;

G₄ is selected from the group consisting of hydrogen, optionally substituted aryl, optionally substituted heteroaryl, optionally substituted cycloalkyl, optionally substituted cycloheteroalkyl, optionally substituted cycloalkenyl, optionally substituted fused aryl, optionally substituted fused heteroaryl, and optionally substituted fused cycloalkyl; and

With the provisos that:

r and s are not both 0;

R₄ is not hydroxy or NH₂ when Y₁ is N;

R₅ is not hydroxy or NH₂ when Y₂ is N;

R₇ and R₈ are not hydroxy or NH₂ when attached to a ring carbon atom adjacent to a ring nitrogen atom;

when G₃ is —(CR₉R₁₀)ₘ—, then none of R₉, R₁₀ and G₄ are selected from the group consisting of unsubstituted phenyl, 4-fluorophenyl, and cyclohexyl;

when G₃ is —(CR₉R₁₀)_{q}J(CR₉R₁₀)ₖ, J is C(O), and q and k are both 0, G₄ is not 2-furanyl; and

when G₄ is said optionally substituted cycloheteroalkyl, said optional substituents are non-cyclic.

In certain embodiments, compounds of the present invention have a structural formula selected from the group consisting of:

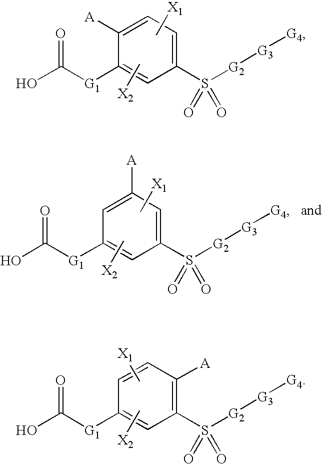

Compounds according to the present invention possess useful PPAR-modulating activity, and may be used in the treatment or prophylaxis of a disease or condition in which PPAR plays an active role. Thus, in broad aspect, the present invention also provides pharmaceutical compositions comprising one or more compounds of the present invention together with a pharmaceutically acceptable carrier, as well as methods of making and using the compounds and compositions. In certain embodiments, the present invention provides methods for modulating PPAR. In other embodiments, the present invention provides methods for treating a PPAR-mediated disorder in a patient in need of such treatment comprising administering to said patient a therapeutically effective amount of a compound or composition according to the present invention. The present invention also contemplates the use of compounds disclosed herein for use in the manufacture of a medicament for the treatment of a disease or condition ameliorated by the modulation of PPAR. In preferred embodiments, the compounds of the invention are modulators of PPARδ.

### DETAILED DESCRIPTION OF THE INVENTION

In certain embodiments, the compounds of the present invention have structural Formula (I) wherein:

G₁ is —(CR₁R₂)ₙ—; and

R₁ and R₂ are each independently selected from the group consisting of hydrogen, halogen, optionally substituted lower alkyl, optionally substituted lower heteroalkyl, or optionally substituted lower alkoxy.

In further embodiments, R₁ and R₂ are each independently selected from the group consisting of hydrogen, methyl, ethyl, and propyl, or together may form a cyclopropyl, cyclobutyl, cyclopentyl, or cyclohexyl.

In yet further embodiments, R₁ and R₂ are hydrogen.

In certain embodiments, T is —C(O)OH.

In yet further embodiments, n=1.

In certain embodiments, A, X₁, and X₂ are each independently selected from the group consisting of hydrogen, optionally substituted lower alkyl, lower perhaloalkyl, and halogen. In further embodiments, A is either hydrogen or methyl and X₁ and X₂ are hydrogen.

In certain embodiments, compounds of Formula I have a structural formula selected from the group consisting of:

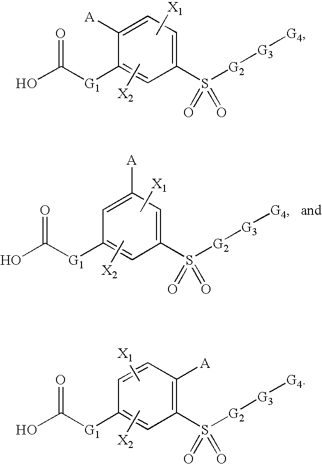

In certain embodiments, A is either hydrogen or methyl and X₁ and X₂ are hydrogen.

In further embodiments, G₃ is —(CR₉R₁₀)ₘ—. In yet further embodiments, R₉ and R₁₀ are hydrogen. In further embodiments, m=1 to 2.

In certain embodiments, G₃ is —(CR₉R₁₀)_{q}J(CR₉R₁₀)ₖ—. In further embodiments, R₉ and R₁₀ are hydrogen. In yet further embodiments, q is 0. In yet other embodiments, J is C(O).

In certain embodiments, G₄ has the structure:

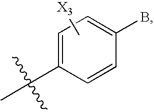

wherein B is selected from the group consisting of hydrogen, halogen, optionally substituted lower alkyl, optionally substituted heteroalkyl, optionally substituted cycloalkyl, optionally substituted heteroalkyl, optionally substituted cycloheteroalkyl, optionally substituted lower alkynyl, optionally substituted lower alkoxy, lower perhaloalkyl, lower perhaloalkoxy, nitro, cyano, hydroxy, NH₂ and —CO₂R₁₁;

R₁₁ is selected from the group consisting of optionally substituted lower alkyl and hydrogen; and

X₃ is selected from the group consisting of hydrogen, halogen, optionally substituted lower alkyl, optionally substituted heteroalkyl, optionally substituted cycloalkyl, optionally substituted cycloheteroalkyl, optionally substituted lower alkynyl, optionally substituted lower alkoxy, lower perhaloalkyl, lower perhaloalkoxy, nitro, cyano, hydroxy, NH₂ and —CO₂R₁₁.

In further embodiments, B is selected from the group consisting of hydrogen, halogen, perhalomethyl, and perhalomethoxy.

In further embodiments, G₃ is —(CR₉R₁₀)ₘ—. In yet further embodiments, R₉ and R₁₀ are hydrogen. In further embodiments, m=1 to 2.

In certain embodiments, G₂ has the structure

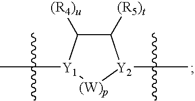

wherein Y₁ and Y₂ are both N;

each W is —CR₇R₈—;

p is 2;

R₄, R₅, R₇, and R₈ are each independently selected from the group consisting of hydrogen, optionally substituted lower alkyl, halogen, lower perhaloalkyl, hydroxy, optionally substituted heteroalkyl, optionally substituted cycloalkyl, optionally substituted lower alkoxy, nitro, cyano, lower perhaloalkoxy, NH₂, and —C(O)—O—R₁₁;

R₁₁ is hydrogen or optionally substituted lower alkyl;

u and t are each 1 or 2; and

with the proviso that at least one of R₄, R₅, R₇, and R₈ is not hydrogen. In further embodiments, said at least one of R₄, R₅, R₇, and R₈ is lower alkyl. In yet further embodiments, said at least one of R₄, R₅, R₇, and R₈ is methyl. In yet further embodiments, at least two of R₄, R₅, R₇, and R₈ are methyl. In yet further embodiments, R₄ and R₇ are methyl and are attached to the piperazine ring at the 2 and 6 positions. In yet further embodiments, R₄ and R₇ methyl groups are oriented cis to each other.

In yet other more preferred embodiments, wherein:

G₃ is —(CR₉R₁₀)ₘ;

R₉ and R₁₀ are hydrogen; and

m is 1 to 2.

In yet other more preferred embodiments, wherein:

G₃ is —(CR₉R₁₀)_{q}J(CR₉R₁₀)ₖ—;

R₉ and R₁₀ are hydrogen;

q is 0; and

J is C(O).

In yet more preferred embodiments, the compounds of the present invention have structural Formula (II) wherein:

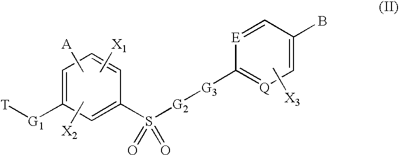

or salt, ester, or prodrug thereof, wherein:

T is —C(O)OH, —C(O)NH₂, or tetrazole;

G₁ is —(CR₁R₂)ₙ—;

n is 1 to 5;

R₁ and R₂ are each independently selected from the group consisting of hydrogen, halogen, optionally substituted lower alkyl, optionally substituted lower heteroalkyl, and optionally substituted lower alkoxy;

A, X₁, and X₁ are each independently selected from the group consisting of hydrogen, optionally substituted lower alkyl, optionally substituted cycloalkyl, halogen, optionally substituted heteroalkyl, optionally substituted cycloheteroalkyl, optionally substituted lower alkynyl, perhaloalkyl, perhaloalkoxy, hydroxy, optionally substituted lower alkoxy, nitro, cyano, and optionally substituted amino;

G₂ is a 5, 6, or 7-membered carbocycle or heterocycle having the structure

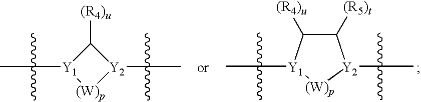

Y₁ and Y₂ are each independently selected from the group consisting of CR₆ and N;

W is selected from the group consisting of —CR₇R₈—, and —CR₇— joined together with Y₁ or Y₂ by a double bond;

p is 1, 2 or 3;

R₄ and R₅ are each independently selected from the group consisting of hydrogen, optionally substituted lower alkyl, halogen, lower perhaloalkyl, hydroxy, optionally substituted heteroalkyl, optionally substituted cycloalkyl, optionally substituted lower alkoxy, nitro, cyano, lower perhaloalkoxy, NH₂, and —C(O)—O—R₁₁; or, when both Y₁ and Y₂ are N, one of W may be taken together with one of R₄ or R₅ to form an optionally substituted 1- or 2-carbon bridge;

R₁₁ is selected from the group consisting of hydrogen and optionally substituted lower alkyl;

R₆ is selected from the group consisting of hydrogen, optionally substituted lower alkyl, hydroxy, and lower perhaloalkyl, or is null when Y₁ or Y₂ is joined to W by a double bond;

u and t are each independently 1 or 2;

R₇ and R₈ are each independently selected from the group consisting of hydrogen, optionally substituted lower alkyl, optionally substituted cycloalkyl, optionally substituted heteroalkyl, hydroxy, optionally substituted lower alkoxy, cyano, halogen, lower perhaloalkyl, NH₂, and a moiety which taken together with R₄ and R₅ forms a 1 or 2 carbon bridge;

G₃ is selected from the group consisting of —(CR₉R₁₀)ₘ—, and —(CR₉R₁₀)_{q}J(CR₉R₁₀)ₖ—;

J is O, S, SO₂, C(O) or NR₁₂;

m is 1 to 3;

q is 0 to 3;

k is 0 to 3;

R₉ and R₁₀ are each independently selected from the group consisting of hydrogen, optionally substituted lower alkyl, optionally substituted lower alkoxy, lower perhaloalkyl, cyano, and nitro;

R₁₂ is selected from the group consisting of hydrogen, optionally substituted lower alkyl, and optionally substituted heteroalkyl;

E and Q are each independently selected from the group consisting of CR₁₃ and N;

each R₁₃ is selected from the group consisting of hydrogen, halogen, optionally substituted lower alkyl, optionally substituted heteroalkyl, optionally substituted cycloalkyl, optionally substituted cycloheteroalkyl, optionally substituted lower alkynyl, optionally substituted lower alkoxy, lower perhaloalkyl, lower perhaloalkoxy, nitro, cyano, hydroxy, NH₂ and —CO₂R₁₁;

X₃ is selected from the group consisting of hydrogen, halogen, optionally substituted lower alkyl, optionally substituted heteroalkyl, optionally substituted cycloalkyl, optionally substituted cycloheteroalkyl, optionally substituted lower alkynyl, optionally substituted lower alkoxy, lower perhaloalkyl, lower perhaloalkoxy, nitro, cyano, hydroxy, NH₂ and —CO₂R₁₁; and

B is selected from the group consisting of optionally substituted lower alkyl, optionally substituted heteroalkyl, optionally substituted cycloalkyl, optionally substituted heteroalkyl, optionally substituted cycloheteroalkyl, optionally substituted lower alkynyl, optionally substituted lower alkoxy, lower perhaloalkyl, lower perhaloalkoxy, nitro, cyano, hydroxy, chloro, bromo, NH₂ and —CO₂R₁₁.

The present invention discloses that compounds of the present invention can modulate at least one peroxisome proliferator-activated receptor (PPAR) function. Compounds described herein may be modulating both PPARδ and PPARγ, or PPARα and PPARδ, or PPARγ and PPARα, or all three PPAR subtypes, or selectively modulating predominantly PPARγ, PPARα or PPARδ. Thus, the present invention provides for a method of modulating PPAR comprising contacting said PPAR with a compound of the invention. In certain embodiments, said modulation is selective for PPARδ over PPARα and PPARγ. In certain embodiments, said modulation of PPARδ is 100-fold selective or greater over said other isoforms. Most preferably, said modulation is 200- to 500-fold selective over said other isoforms. In preferred embodiments, compounds of the present invention are also selective for PPAR over other nuclear receptors and proteins, such as, for example, GPR40.

Thus, one aspect of the present invention discloses a method of modulating at least one peroxisome proliferator-activated receptor (PPAR) function comprising the step of contacting the PPAR with a compound of Formula I, as described herein. The change in cell phenotype, cell proliferation, activity of the PPAR, expression of the PPAR or binding of the PPAR with a natural binding partner may be monitored. Such methods may be modes of treatment of disease, biological assays, cellular assays, biochemical assays, or the like. In preferred embodiments, compounds the present invention have EC₅₀ values less than 5 μM against PPAR as measured by functional cell assay. In further preferred embodiments, said compounds have EC₅₀ values less than 5 μM against PPARδ.

In another aspect, the present invention discloses methods of treatment of a PPAR-mediated disease comprising the administration of a therapeutically effective amount of a compound of Formula I or a pharmaceutically acceptable salt, ester, amide, or prodrug thereof, to a patient in need thereof. In certain embodiments of this aspect, the present invention discloses methods: for raising HDL, lowering LDLc, shifting LDL particle size from small dense to normal LDL, inhibiting cholesterol absorption, or reducing triglycerides, in a subject; for decreasing insulin resistance or lowering blood pressure in a subject; for treating obesity, diabetes, especially Type 2 diabetes, hyperinsulinemia, metabolic syndrome X, dyslipidemia, and hypercholesterolemia; for treating cardiovascular diseases including vascular disease, atherosclerosis, coronary heart disease, cerebrovascular disease, heart failure and peripheral vessel disease in a subject; for treating hyperproliferative disorders including cancers such as colon, skin, and lung cancers in a subject; for treating inflammatory diseases, including asthma, rheumatoid arthritis, osteoarthritis, disorders associated with oxidative stress, inflammatory response to tissue injury, psoriasis, ulcerative colitis, dermatitis, and autoimmune disease in a subject; for treating opthalmologic diseases including dry eye (including Sjögren's syndrome), macular degeneration, closed and wide angle glaucoma, inflammation, and pain of the eye; for treating polycystic ovary syndrome, climacteric, pathogenesis of emphysema, ischemia-associated organ injury, doxorubicin-induced cardiac injury, drug-induced hepatotoxicity, hypertoxic lung injury, scarring, wound healing, anorexia nervosa and bulimia nervosa in a subject; for upregulating the expression of GLUT4 in adipose tissue in a subject; or for reducing the expression of NPC1 L1 in a subject; all comprising the administration of a therapeutic amount of a compound of Formula I to a patient in need thereof. Preferably, the PPAR may be selected from the group consisting of PPARα, PPARδ, and PPARγ. More preferably, the PPAR is PPARδ.

Another aspect of the invention are pharmaceutical compositions comprising compounds of Formula I together with pharmaceutically acceptable diluents or carriers. Thus, the present invention provides for compounds of Formula I or pharmaceutical compositions thereof for use in the treatment of a disease or condition ameliorated by the modulation of a PPAR.

In yet another aspect, the invention further discloses compounds of Formula I or pharmaceutical compositions thereof for use in the manufacture of a medicament for the prevention or treatment of a disease or condition ameliorated by the modulation of a PPAR. The invention also discloses the use of a compound of Formula I according to the invention for the manufacture of a medicament: for raising HDL, lowering LDLc, shifting LDL particle size from small dense to normal LDL, inhibiting cholesterol absorption, or reducing triglycerides, in a subject; for decreasing insulin resistance or lowering blood pressure in a subject; for treating obesity, diabetes, especially Type 2 diabetes, hyperinsulinemia, metabolic syndrome X, dyslipidemia, and hypercholesterolemia; for treating cardiovascular diseases including vascular disease, atherosclerosis, coronary heart disease, cerebrovascular disease, heart failure and peripheral vessel disease in a subject; for treating hyperproliferative disorders including cancers such as colon, skin, and lung cancers in a subject; for treating inflammatory diseases, including asthma, rheumatoid arthritis, osteoarthritis, disorders associated with oxidative stress, inflammatory response to tissue injury, psoriasis, ulcerative colitis, dermatitis, and autoimmune disease in a subject; for treating opthalmologic diseases including dry eye (including Sjögren's syndrome), macular degeneration, closed and wide angle glaucoma, inflammation, and pain of the eye; for treating polycystic ovary syndrome, climacteric, pathogenesis of emphysema, ischemia-associated organ injury, doxorubicin-induced cardiac injury, drug-induced hepatotoxicity, hypertoxic lung injury, scarring, wound healing, anorexia nervosa and bulimia nervosa in a subject; for upregulating the expression of GLUT4 in adipose tissue in a subject; or for reducing the expression of NPC1L1 in a subject; all comprising the administration of a therapeutic amount of a compound of Formula I to a patient in need thereof. Preferably, the PPAR may be selected from the group consisting of PPARα, PPARδ, and PPARγ. More preferably, the PPAR is PPARδ.

As used herein, the following terms have the meanings indicated.

The term “acyl,” as used herein, alone or in combination, refers to a carbonyl attached to an alkenyl, allyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl. Examples of acyl groups include formyl, alkanoyl and aroyl.

The term “alkenyl,” as used herein, alone or in combination, refers to a straight-chain or branched-chain hydrocarbon radical having one or more double bonds and containing from 2 to 20, preferably 2 to 6, carbon atoms. Alkenylene refers to a carbon-carbon double bond system attached at two or more positions such as ethenylene [(—CH═CH—),(—C::C—)]. Examples of suitable alkenyl radicals include ethenyl, propenyl, 2-methylpropenyl, 1,4-butadienyl and the like.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups may be optionally substituted as defined herein. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein, alone or in combination, refers to an alkyl group attached to the parent molecular moiety through an amino group. Suitable alkylamino groups may be mono- or dialkylated, forming groups such as, for example, N-methylamino, N-ethylamino, N,N-dimethylamino, N,N-ethylmethylamino and the like.

The term “alkylidene,” as used herein, alone or in combination, refers to an alkenyl group in which one carbon atom of the carbon-carbon double bond belongs to the moiety to which the alkenyl group is attached.

The term “alkylthio,” as used herein, alone or in combination, refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like.

The term “alkynyl,” as used herein, alone or in combination, refers to a straight-chain or branched chain hydrocarbon radical having one or more triple bonds and containing from 2 to 20, preferably from 2 to 6, more preferably from 2 to 4, carbon atoms. “Alkynylene” refers to a carbon-carbon triple bond attached at two positions such as ethynylene (—C [symbol] C—, —C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like.

The terms “amido” and “carbamoyl,” as used herein, alone or in combination, refer to an amino group as described below attached to the parent molecular moiety through a carbonyl group, or vice versa. The term “C-amido” as used herein, alone or in combination, refers to a —C(═O)—NR₂ group with R as defined herein. The term “N-amido” as used herein, alone or in combination, refers to a RC(═O)NH— group, with R as defined herein. The term “acylamino” as used herein, alone or in combination, embraces an acyl group attached to the parent moiety through an amino group. An example of an “acylamino” group is acetylamino (CH₃C(O)NH—).

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which may themselves be optionally substituted.

The term “aryl,” as used herein, alone or in combination, means a carbocyclic aromatic system containing one, two or three rings wherein such rings may be attached together in a pendent manner or may be fused. The term “aryl” embraces aromatic radicals such as benzyl, phenyl, naphthyl, anthracenyl, phenanthryl, indanyl, indenyl, annulenyl, azulenyl, tetrahydronaphthyl, and biphenyl.

The term “arylalkenyl” or “aralkenyl,” as used herein, alone or in combination, refers to an aryl group attached to the parent molecular moiety through an alkenyl group.

The term “arylalkoxy” or “aralkoxy,” as used herein, alone or in combination, refers to an aryl group attached to the parent molecular moiety through an alkoxy group.

The term “arylalkyl” or “aralkyl,” as used herein, alone or in combination, refers to an aryl group attached to the parent molecular moiety through an alkyl group.

The term “arylalkynyl” or “aralkynyl,” as used herein, alone or in combination, refers to an aryl group attached to the parent molecular moiety through an alkynyl group.

The term “arylalkanoyl” or “aralkanoyl” or “aroyl,” as used herein, alone or in combination, refers to an acyl radical derived from an aryl-substituted alkanecarboxylic acid such as benzoyl, napthoyl, phenylacetyl, 3-phenylpropionyl (hydrocinnamoyl), 4-phenylbutyryl, (2-naphthyl)acetyl, 4-chlorohydrocinnamoyl, and the like.

The term aryloxy as used herein, alone or in combination, refers to an aryl group attached to the parent molecular moiety through an oxy.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which may be attached to the parent molecular moiety from either the nitrogen or acid end, and which may be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O)NR′— group, with R and R′ as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cyano,” as used herein, alone or in combination, refers to —CN.

The term “cycloalkyl,” or, alternatively, “carbocycle,” as used herein, alone or in combination, refers to a saturated or partially saturated monocyclic, bicyclic or tricyclic alkyl radical wherein each cyclic moiety contains from 3 to 12, preferably five to seven, carbon atom ring members and which may optionally be a benzo fused ring system which is optionally substituted as defined herein. Examples of such cycloalkyl radicals include cyclopropyl, cyclobutyl, cyclopentyl, cyclohexyl, cycloheptyl, octahydronaphthyl, 2,3-dihydro-1H-indenyl, adamantyl and the like. “Bicyclic” and “tricyclic” as used herein are intended to include both fused ring systems, such as decahydronapthalene, octahydronapthalene as well as the multicyclic (multicentered) saturated or partially unsaturated type. The latter type of isomer is exemplified in general by, bicyclo[1,1,1]pentane, camphor, adamantane, and bicyclo[3,2,1]octane.

The term “ester,” as used herein, alone or in combination, refers to a carboxy group bridging two moieties linked at carbon atoms.

The term “ether,” as used herein, alone or in combination, refers to an oxy group bridging two moieties linked at carbon atoms.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms may optionally be oxidized and the nitrogen heteroatom may optionally be quaternized. The heteroatom(s) O, N and S may be placed at any interior position of the heteroalkyl group. Up to two heteroatoms may be consecutive, such as, for example, —CH₂—NH—OCH₃.

The term “heteroaryl,” as used herein, alone or in combination, refers to 3 to 7 membered, preferably 5 to 7 membered, unsaturated heteromonocyclic rings, or fused polycyclic rings in which at least one of the fused rings is unsaturated, wherein at least one atom is selected from the group consisting of O, S, and N. The term also embraces fused polycyclic groups wherein heterocyclic radicals are fused with aryl radicals, wherein heteroaryl radicals are fused with other heteroaryl radicals, or wherein heteroaryl radicals are fused with cycloalkyl radicals. Examples of heteroaryl groups include pyrrolyl, pyrrolinyl, imidazolyl, pyrazolyl, pyridyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom may be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the invention are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited.

The term “hydrazinyl” as used herein, alone or in combination, refers to two amino groups joined by a single bond, i.e., —N—N—.

The term “hydroxy,” as used herein, alone or in combination, refers to —OH.

The term “hydroxyalkyl,” as used herein, alone or in combination, refers to a hydroxy group attached to the parent molecular moiety through an alkyl group.

The term “imino,” as used herein, alone or in combination, refers to ═N—.

The term “iminohydroxy,” as used herein, alone or in combination, refers to ═N(OH) and ═N—O—.

The phrase “in the main chain” refers to the longest contiguous or adjacent chain of carbon atoms starting at the point of attachment of a group to the compounds of this invention.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “lower,” as used herein, alone or in combination, means containing from 1 to and including 6 carbon atoms.

The term “mercaptyl” as used herein, alone or in combination, refers to an RS— group, where R is as defined herein.

The term “nitro,” as used herein, alone or in combination, refers to —NO₂.

The terms “oxy” or “oxa,” as used herein, alone or in combination, refer to —O—.

The term “oxo,” as used herein, alone or in combination, refers to ═O.

The term “perhaloalkoxy” refers to an alkoxy group where all of the hydrogen atoms are replaced by halogen atoms.

The term “perhaloalkyl” as used herein, alone or in combination, refers to an alkyl group where all of the hydrogen atoms are replaced by halogen atoms.

The terms “sulfonate,” “sulfonic acid,” and “sulfonic,” as used herein, alone or in combination, refer the —SO₃H group and its anion as the sulfonic acid is used in salt formation.

The term “sulfanyl,” as used herein, alone or in combination, refers to —S—.

The term “sulfinyl,” as used herein, alone or in combination, refers to —S(O)—.

The term “sulfonyl,” as used herein, alone or in combination, refers to —S(O)₂—.

The term “N-sulfonamido” refers to a RS(═O)₂NR′— group with R and R′ as defined herein.

The term “S-sulfonamido” refers to a —S(═O)₂NRR′, group, with R and R′ as defined herein.

The terms “thia” and “thio,” as used herein, alone or in combination, refer to a —S— group or an ether wherein the oxygen is replaced with sulfur. The oxidized derivatives of the thio group, namely sulfinyl and sulfonyl, are included in the definition of thia and thio.

The term “thiol,” as used herein, alone or in combination, refers to an —SH group.

The term “thiocarbonyl,” as used herein, when alone includes thioformyl —C(S)H and in combination is a —C(S)— group.

The term “N-thiocarbamyl” refers to an ROC(S)NR′— group, with R and R′ as defined herein.

The term “O-thiocarbamyl” refers to a —OC(S)NRR′, group with R and R′as defined herein.

The term “thiocyanato” refers to a —CNS group.

The term “trihalomethanesulfonamido” refers to a X₃CS(O)₂NR— group with X is a halogen and R as defined herein.

The term “trihalomethanesulfonyl” refers to a X₃CS(O)₂— group where X is a halogen.

The term “trihalomethoxy” refers to a X₃CO— group where X is a halogen.

The term “trisubstituted silyl,” as used herein, alone or in combination, refers to a silicone group substituted at its three free valences with groups as listed herein under the definition of substituted amino. Examples include trimethysilyl, tert-butyldimethylsilyl, triphenylsilyl and the like.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When a group is defined to be “null,” what is meant is that said group is absent.

The term “optionally substituted” means the anteceding group may be substituted or unsubstituted. When substituted, the substituents of an “optionally substituted” group may include, without limitation, one or more substituents independently selected from the following groups or a particular designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents may be joined together to form a fused five-, six-, or seven-membered carbocyclic or heterocyclic ring consisting of zero to three heteroatoms, for example forming methylenedioxy or ethylenedioxy. An optionally substituted group may be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a particular moiety may be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which may be optionally substituted. Such R and R′ groups should be understood to be optionally substituted as defined herein. Whether an R group has a number designation or not, every R group, including R, R′ and R″ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups may be attached to a parent molecule or may occupy a position in a chain of elements from either end as written. Thus, by way of example only, an unsymmetrical group such as —C(O)N(R)— may be attached to the parent moiety at either the carbon or the nitrogen.

Asymmetric centers exist in the compounds of the present invention. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention. Additionally, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “activate” refers to increasing the cellular function of a PPAR.

The term “inhibit” refers to decreasing the cellular function of a PPAR. The PPAR function may be the interaction with a natural binding partner or catalytic activity.

The term “modulate” refers to the ability of a compound of the invention to alter the function of a PPAR. A modulator may activate the activity of a PPAR. The term “modulate” also refers to altering the function of a PPAR by increasing or decreasing the probability that a complex forms between a PPAR and a natural binding partner. A modulator may increase the probability that such a complex forms between the PPAR and the natural binding partner, may increase or decrease the probability that a complex forms between the PPAR and the natural binding partner depending on the concentration of the compound exposed to the PPAR, and or may decrease the probability that a complex forms between the PPAR and the natural binding partner.

“PPAR modulator” is used herein to refer to a compound that exhibits an EC₅₀ with respect to PPAR activity of no more than about 100 μM and more typically not more than about 50 μM, as measured in the PPAR assay described generally hereinbelow. “EC₅₀” is that concentration of modulator which either activates or reduces the activity of an enzyme (e.g., PPAR) to half-maximal level. Representative compounds of the present invention have been discovered to exhibit modulatory activity against PPAR. Compounds of the present invention preferably exhibit an EC₅₀ with respect to PPAR of no more than about 10 μM, more preferably, no more than about 5 μM, even more preferably not more than about 1 μM, and most preferably, not more than about 200 nM, as measured in the PPAR assay described herein.

The term “selective” as used herein means having the characteristic or property of being highly specific in binding, activity, or effect. Compounds described herein as selective for one PPAR isoform over another may preferentially bind one isoform of PPAR, for example PPARδ, over another, such as PPARα. Compounds described herein as selective for PPAR over GPR40 may preferentially bind and/or modulate PPAR in favor of GPR40. The degree of selectivity may vary, but preferably a selective compound would be at least tenfold selective for the desired target (e.g., PPAR). More preferably, the compound would be 100- to 1000-fold selective. Alternatively, a compound may be selective the sense of producing a differential effect. For example, such a compound may bind both PPAR and GPR40 with equal or similar affinity, but activate one while inhibiting the other.

The phrase “therapeutically effective” is intended to qualify the amount of active ingredients used in the treatment of a disease or disorder. This amount will achieve the goal of reducing or eliminating the said disease or disorder. In reference to the treatment of diabetes or dyslipidemia, for example, a therapeutically effective amount may refer to that amount which has the effect of (1) reducing the blood glucose levels; (2) normalizing lipids, e.g. triglycerides, low-density lipoprotein; (3) relieving to some extent (or, preferably, eliminating) one or more symptoms associated with the disease, condition or disorder to be treated; and/or (4) raising HDL.

As used herein, reference to “treatment” of a patient is intended to include prophylaxis. The term “patient” means all mammals including humans. Examples of patients include humans, cows, dogs, cats, goats, sheep, pigs, and rabbits. Preferably, the patient is a human.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

The term “prodrug” refers to a compound that is made more active in vivo. The present compounds can also exist as prodrugs, as described in Hydrolysis in Drug and Prodrug Metabolism: Chemistry, Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment. For example, prodrugs can be slowly converted to a compound when placed in a transdermal patch reservoir with a suitable enzyme or chemical reagent. Prodrugs are often useful because, in some situations, they may be easier to administer than the compound, or parent drug. They may, for instance, be bioavailable by oral administration whereas the parent drug is not. The prodrug may also have improved solubility in pharmaceutical compositions over the parent drug. A wide variety of prodrug derivatives are known in the art, such as those that rely on hydrolytic cleavage or oxidative activation of the prodrug. An example, without limitation, of a prodrug would be a compound which is administered as an ester (the “prodrug”), but then is metabolically hydrolyzed to the carboxylic acid, the active entity. Additional examples include peptidyl derivatives of a compound. The term “therapeutically acceptable prodrug,” refers to those prodrugs or zwitterions which are suitable for use in contact with the tissues of patients without undue toxicity, irritation, and allergic response, are commensurate with a reasonable benefit/risk ratio, and are effective for their intended use.

The term “therapeutically acceptable salt,” as used herein, represents salts or zwitterionic forms of the compounds of the present invention which are water or oil-soluble or dispersible; which are suitable for treatment of diseases without undue toxicity, irritation, and allergic-response; which are commensurate with a reasonable benefit/risk ratio; and which are effective for their intended use. The salts can be prepared during the final isolation and purification of the compounds or separately by reacting the appropriate compound in the form of the free base with a suitable acid.

Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds of the present invention can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds of the compounds of the present invention and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

The compounds of the present invention can exist as therapeutically acceptable salts. The present invention includes compounds listed above in the form of salts, in particular acid addition salts.

Suitable salts include those formed with both organic and inorganic acids. Such acid addition salts will normally be pharmaceutically acceptable. However, salts of non-pharmaceutically acceptable salts may be of utility in the preparation and purification of the compound in question. For a more complete discussion of the preparation and selection of salts, refer to Pharmaceutical Salts: Properties, Selection, and Use (Stahl, P. Heinrich. Wiley-VCHA, Zurich, Switzerland, 2002).

Thus, salts include the hydrochloride, hydrobromide, sulfonate, citrate, tartrate, phosphonate, lactate, pyruvate, acetate, succinate, oxalate, fumarate, malate, oxaloacetate, methanesulfonate, ethanesulfonate, p-toluenesulfonate, benzenesulfonate and isethionate salts of the compounds of the present invention. The salts can be prepared by contacting the compounds of the invention with an appropriate acid, either neat or in a suitable inert solvent, to yield the salt forms of the invention. In certain embodiments, the p-toluenesulfonate (tosylate) is used with the disclosed compounds.

While it may be possible for the compounds of the subject invention to be administered as the raw chemical, it is also possible to present them as a pharmaceutical formulation. Accordingly, the subject invention provides a pharmaceutical formulation comprising a compound or a pharmaceutically acceptable salt, ester, prodrug or solvate thereof, together with one or more pharmaceutically acceptable carriers thereof and optionally one or more other therapeutic ingredients. The carrier(s) must be “acceptable” in the sense of being compatible with the other ingredients of the formulation and not deleterious to the recipient thereof. Proper formulation is dependent upon the route of administration chosen. Any of the well-known techniques, carriers, and excipients may be used as suitable and as understood in the art; e.g., in Remington's Pharmaceutical Sciences. The pharmaceutical compositions of the present invention may be manufactured in a manner that is itself known, e.g., by means of conventional mixing, dissolving, granulating, dragee-making, levigating, emulsifying, encapsulating, entrapping or compression processes.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. All methods include the step of bringing into association a compound of the subject invention or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical preparations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the compositions may take the form of tablets, lozenges, pastilles, or gels formulated in conventional manner. Such compositions may comprise the active ingredient in a flavored basis such as sucrose and acacia or tragacanth.

The compounds may also be formulated in rectal compositions such as suppositories or retention enemas, e.g., containing conventional suppository bases such as cocoa butter, polyethylene glycol, or other glycerides.

Compounds of the present invention may be administered topically, that is by non-systemic administration. This includes the application of a compound of the present invention externally to the epidermis or the buccal cavity and the instillation of such a compound into the ear, eye and nose, such that the compound does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient may comprise, for topical administration, from 0.001% to 10% w/w, for instance from 1% to 2% by weight of the formulation. It may however comprise as much as 10% w/w but preferably will comprise less than 5% w/w, more preferably from 0.1% to 1% w/w of the formulation.

Gels for topical or transdermal administration of compounds of the subject invention may comprise, generally, a mixture of volatile solvents, nonvolatile solvents, and water. The volatile solvent component of the buffered solvent system may preferably include lower (C1-C6) alkyl alcohols, lower alkyl glycols and lower glycol polymers. More preferably, the volatile solvent is ethanol. The volatile solvent component is thought to act as a penetration enhancer, while also producing a cooling effect on the skin as it evaporates. The nonvolatile solvent portion of the buffered solvent system is selected from lower alkylene glycols and lower glycol polymers. Preferably, propylene glycol is used. The nonvolatile solvent slows the evaporation of the volatile solvent and reduces the vapor pressure of the buffered solvent system. The amount of this nonvolatile solvent component, as with the volatile solvent, is determined by the pharmaceutical compound or drug being used. When too little of the nonvolatile solvent is in the system, the pharmaceutical compound may crystallize due to evaporation of volatile solvent, while an excess will result in a lack of bioavailability due to poor release of drug from solvent mixture. The buffer component of the buffered solvent system may be selected from any buffer commonly used in the art; preferably, water is used. The preferred ratio of ingredients is about 20% of the nonvolatile solvent, about 40% of the volatile solvent, and about 40% water. There are several optional ingredients which can be added to the topical composition. These include, but are not limited to, chelators and gelling agents. Appropriate gelling agents can include, but are not limited to, semisynthetic cellulose derivatives (such as hydroxypropylmethylcellulose) and synthetic polymers, and cosmetic agents.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy base. The base may comprise hydrocarbons such as hard, soft or liquid paraffin, glycerol, beeswax, a metallic soap; a mucilage; an oil of natural origin such as almond, corn, arachis, castor or olive oil; wool fat or its derivatives or a fatty acid such as steric or oleic acid together with an alcohol such as propylene glycol or a macrogel. The formulation may incorporate any suitable surface active agent such as an anionic, cationic or non-ionic surfactant such as a sorbitan ester or a polyoxyethylene derivative thereof. Suspending agents such as natural gums, cellulose derivatives or inorganic materials such as silicaceous silicas, and other ingredients such as lanolin, may also be included.

Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified by filtration, transferred to a suitable container which is then sealed and sterilized by autoclaving or maintaining at 98-100° C. for half an hour. Alternatively, the solution may be sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

For administration by inhalation the compounds according to the invention are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds according to the invention may take the form of a dry powder composition, for example a powder mix of the compound and a suitable powder base such as lactose or starch. The powder composition may be presented in unit dosage form, in for example, capsules, cartridges, gelatin or blister packs from which the powder may be administered with the aid of an inhalator or insufflator.

Preferred unit dosage formulations are those containing an effective dose, as herein below recited, or an appropriate fraction thereof, of the active ingredient.

It should be understood that in addition to the ingredients particularly mentioned above, the formulations of this invention may include other agents conventional in the art having regard to the type of formulation in question, for example those suitable for oral administration may include flavoring agents.

The compounds of the invention may be administered orally or via injection at a dose of from 0.1 to 500 mg/kg per day. The dose range for adult humans is generally from 5 mg to 2 g/day. Tablets or other forms of presentation provided in discrete units may conveniently contain an amount of compound of the invention which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds of the subject invention can be administered in various modes, e.g. orally, topically, or by injection. The precise amount of compound administered to a patient will be the responsibility of the attendant physician. The specific dose level for any particular patient will depend upon a variety of factors including the activity of the specific compound employed, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt, ester, or prodrug thereof) in combination with another therapeutic agent. By way of example only, if one of the side effects experienced by a patient upon receiving one of the compounds herein is hypertension, then it may be appropriate to administer an anti-hypertensive agent in combination with the initial therapeutic agent. Or, by way of example only, the therapeutic effectiveness of one of the compounds described herein may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein with another therapeutic agent (which also includes a therapeutic regimen) that also has therapeutic benefit. By way of example only, in a treatment for diabetes involving administration of one of the compounds described herein, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of the compound of formula (I) with: (a) statin and/or other lipid lowering drugs for example MTP inhibitors and LDLR upregulators; (b) antidiabetic agents, including fibrates (such as metformin), sulfonylureas, or PPAR-gamma, PPAR-alpha and PPAR-alpha/gamma modulators including thiazolidinediones (such as pioglitazone and rosiglitazone); (c) antihypertensive agents such as angiotensin antagonists, (such as telmisartan), calcium channel antagonists (such as lacidipine), and acetylcholinesterase (ACE) inhibitors, such as enalapril; and (d) anti-inflammatory agents including nonsteroidal anti-inflammatory agents such as cyclooxygenase 1 (COX-1) and/or COX-2 inhibitors (both selective and nonselective agents including ibuprofen and celecoxib).

For the treatment of opthalmologic disorders and diseases of the eye, compounds according to the present invention may be administered with an agent selected from the group comprising: nitric oxide synthase inhibitors including inhibitors of inducible nitric oxide synthase; inhibitors of p38 kinase; beta-blockers including timolol, betaxolol, levobetaxolol, carteolol, levobunolol, and propranolol; carbonic anhydrase inhibitors including brinzolamide and dorzolamide; α- and β-adrenergic antagonists including α1-adrenergic antagonists such as nipradilol and α2 agonists such as iopidine and brimonidine; miotics including pilocarpine and epinephrine; prostaglandin analogs including latanoprost, travoprost, bimatoprost, and unoprostone; corticosteroids including dexamethasone, prednisone, and methylprednisolone; and immunosuppressant agents including azathioprine, cyclosporine, and immunoglobulins.

In any case, the multiple therapeutic agents (at least one of which is a compound of the present invention) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Thus, in another aspect, the present invention provides methods for treating PPAR-mediated disorders in a human or animal subject in need of such treatment comprising administering to said subject an amount of a compound of the present invention effective to reduce or prevent said disorder in the subject in combination with at least one additional agent for the treatment of said disorder that is known in the art. In a related aspect, the present invention provides therapeutic compositions comprising at least one compound of the present invention in combination with one or more additional agents for the treatment of PPAR-mediated disorders.

Besides being useful for human treatment, the compounds and formulations of the present invention are also useful for veterinary treatment of companion animals, exotic animals and farm animals, including mammals, rodents, and the like. More preferred animals include horses, dogs, and cats.

All references, patents or applications, U.S. or foreign, cited in the application are hereby incorporated by reference as if written herein.

### General Synthetic Methods for Preparing Compounds

The following schemes can be used to practice the present invention.

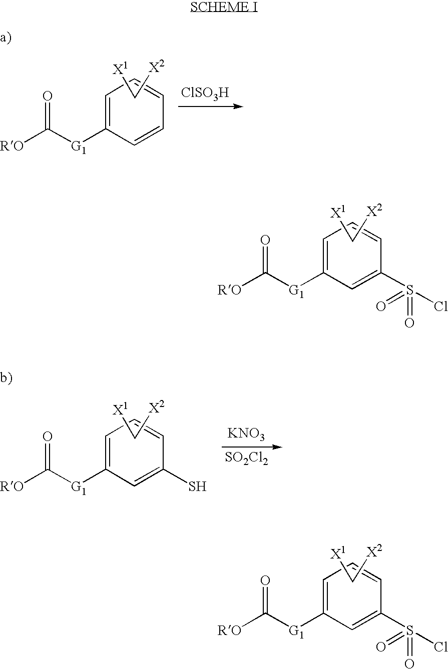

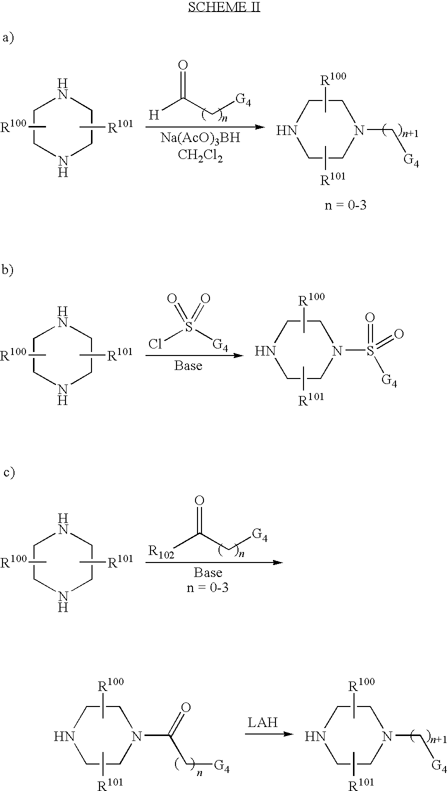

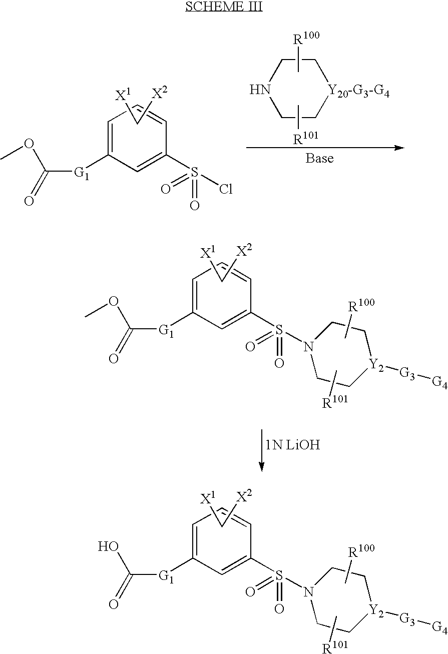

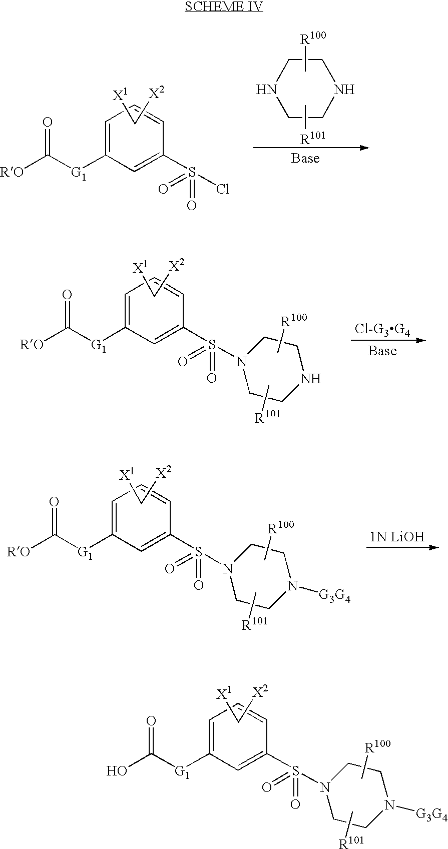

The invention is further illustrated by the following examples.

### EXAMPLE 1

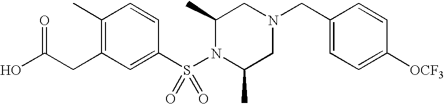

{5-[2,6-Dimethyl-4-(4-trifluoromethoxy-benzyl)-piperazine-1-sulfonyl]-2-methyl-phenyl}-acetic acid:

### Step 1

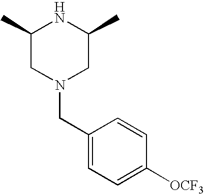

3,5-Dimethyl-1-(4-trifluoromethoxy-benzyl)-piperazine: To a solution of 4-(trifluoromethoxy)-benzaldehyde (776 μL, 4.38 mmol) in dichloromethane (30 mL) was added 2,6-dimethyl piperazine (1.0 g, 8.77 mmol). The reaction mixture was stirred for 1 h. Sodium triacetoxy borohydride (2.45 g, 8.77 mmol) was added and the reaction mixture was stirred for 4 h. The reaction mixture was concentrated in vacuo, diluted with ethyl acetate and extracted with 1N HCl (2×50 mL). The combined aqueous solution was neutralized with NaOH and extracted with ethyl acetate (3×50 mL). The combined organic solution was dried (Na₂SO₄) and concentrated in vacuo to provide 3,5-dimethyl-1-(4-trifluoromethoxy-benzyl)-piperazine (1.01 g, 80%) as a clear oil. ¹H NMR (400 MHz, CD₃OD) δ 7.42 (d, 2H), 7.23 (d, 2H), 3.54 (s, 2H), 2.98-2.88 (m, 2H), 2.82-2.74 (m, 2H), 1.69 (t, 2H), 1.05 (d, 6H); LCMS 289.5 (M+1)⁺.

### Step 2

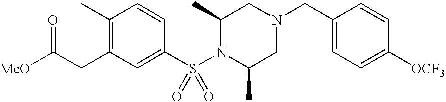

{5-[2,6-Dimethyl-4-(4-trifluoromethoxy-benzyl)-piperazine-1-sulfonyl]-2-methyl-phenyl}-acetic acid methyl ester: To a solution of 3,5-dimethyl-1-(4-trifluoromethoxy-benzyl)-piperazine (50 mg, 0.17 mmol) in acetonitrile (2 mL) was added (5-chlorosulfonyl-2-methyl-phenyl)-acetic acid methyl ester (45 mg, 0.17 mmol) and K₂CO₃ (100 mg, 0.41 mmol). The reaction mixture was heated to 50° C. for 20 h. The reaction mixture was concentrated in vacuo and the residue was purified by chromatography on silica (3:7 EtOAc:hexane) to give {5-[2,6-dimethyl-4-(4-trifluoromethoxy-benzyl)-piperazine-1-sulfonyl]-2-methyl-phenyl}-acetic acid methyl ester (45 mg, 50%). ¹H NMR (400 MHz, CD₃OD) δ 7.72 (s, 1H), 7.63-7.59 (m, 1H), 7.57-7.53 (m, 2H), 7.34-7.29 (m, 3H), 4.42-4.34 (m, 2H), 4.24-4.14 (m, 2H), 3.74 (s, 3H), 3.70 (s, 2H), 3.21-3.13 (m, 2H), 2.58-2.48 (m, 2H), 2.35 (s, 3H), 1.50 (d, 6H); LCMS 514.5 (M+1)⁺.

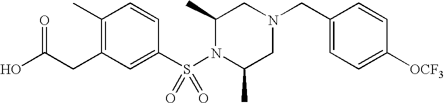

### Step 3

{5-[2,6-Dimethyl-4-(4-trifluoromethoxy-benzyl)-piperazine-1-sulfonyl]-2-methyl-phenyl}-acetic acid: To a solution of {5-[2,6-dimethyl-4-(4-trifluoromethoxy-benzyl)-piperazine-1-sulfonyl]-2-methyl-phenyl}-acetic acid methyl ester (10 mg, 0.02 mmol) in THF (1 mL) and methanol (0.5 mL) was added a 1N solution of LiOH (1 mL). The reaction mixture was stirred at room temperature for 3 h. The reaction mixture was concentrated in vacuo and the residue partitioned between EtOAc and water. The aqueous layer was treated with 1N HCl and extracted with ethyl acetate. The organic layer was dried and concentrated in vacuo. The residue was purified by chromatography on silica (9:1 DCM:MeOH) to give {5-[2,6-dimethyl-4-(4-trifluoromethoxy-benzyl)-piperazine-1-sulfonyl]-2-methyl-phenyl}-acetic acid (5 mg, 50%). ¹H NMR (400 MHz, CD₃OD) δ 7.72 (s, 1H), 7.66-7.60 (m, 1H), 7.59-7.54 (m, 2H), 7.37-7.30 (m, 3H), 4.46-4.38 (m, 2H), 4.26-4.18 (m, 2H), 3.78 (s, 2H), 3.24-3.12 (m, 2H), 2.60-2.50 (m, 2H), 2.38 (s, 3H), 1.49 (d, 6H); LCMS 500.9 (M+1)⁺.

### EXAMPLE 2

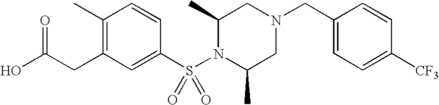

{5-[2,6-Dimethyl-4-(4-trifluoromethyl-benzyl)-piperazine-1-sulfonyl]-2-methyl-phenyl}-acetic acid: The compound {5-[2,6-dimethyl-4-(4-trifluoromethyl-benzyl)-piperazine-1-sulfonyl]-2-methyl-phenyl}-acetic acid was synthesized according to the procedure outlined in Example 1 using 4-(trifluoromethyl)-benzaldehyde. ¹H NMR (400 MHz, CD₃OD) δ 7.74-7.60 (m, 6H), 7.35 (d, 1H), 4.38-4.24 (m, 2H), 4.18-4.01 (m, 2H), 3.77 (s, 2H), 3.18-2.92 (m, 2H), 2.44-2.38 (m, 5H), 1.48 (d, 6H); LCMS 485.5 (M+1)⁺.

### EXAMPLE 3

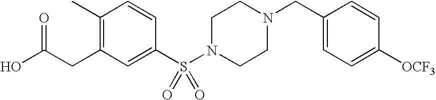

{2-Methyl-5-[4-(4-trifluoromethoxy-benzyl)-piperazine-1-sulfonyl]-phenyl}-acetic acid: The compound {2-methyl-5-[4-(4-trifluoromethoxy-benzyl)-piperazine-1-sulfonyl]-phenyl}-acetic acid was synthesized according to the procedure outlined in Example 1 using piperazine. ¹H NMR (400 MHz, CD₃OD) δ 7.68-7.60 (m, 4H), 7.48-7.43 (m, 1H), 7.42-7.35 (m, 2H), 4.38 (s, 2H), 3.78 (s, 2H), 3.42-3.24 (m, 8H), 2.39 (s, 3H); LCMS 474.0 (M+1)⁺.

### EXAMPLE 4

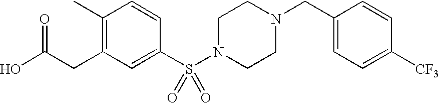

{2-Methyl-5-[4-(4-trifluoromethyl-benzyl)-piperazine-1-sulfonyl]-phenyl}-acetic acid: The compound {2-methyl-5-[4-(4-trifluoromethyl-benzyl)-piperazine-1-sulfonyl]-phenyl}-acetic acid was synthesized according to the procedure outlined in Example 1 using piperazine and 4-(trifluoromethyl)-benzaldehyde. ¹H NMR (400 MHz, CD₃OD) δ 7.82-7.76 (m, 2H), 7.75-7.70 (m, 2H), 7.68-7.65 (m, 1H), 7.64-7.60 (m, 1H), 7.48-7.43 (m, 1H), 4.45 (s, 2H), 3.78 (s, 2H), 3.44-3.22 (m, 8H), 2.39 (s, 3H); LCMS 458.0 (M+1)⁺.

### EXAMPLE 5

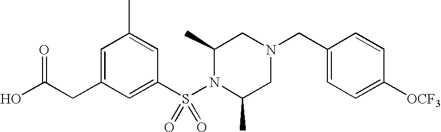

{3-[2,6-Dimethyl-4-(4-trifluoromethoxy-benzyl)-piperazine-1-sulfonyl]-5-methyl-phenyl}-acetic acid: The compound {3-[2,6-dimethyl-4-(4-trifluoromethoxy-benzyl)-piperazine-1-sulfonyl]-5-methyl-phenyl}-acetic acid was synthesized according to the procedure outlined in Example 1 using (3-chlorosulfonyl-5-methyl-phenyl)-acetic acid methyl ester. ¹H NMR (400 MHz, CD₃OD) δ 7.72 (s, 1H), 7.66-7.54 (m, 4H), 7.40-7.30 (m, 3H), 4.52-4.40 (m, 2H), 4.34 (s, 2H), 3.72 (s, 2H), 3.32-3.20 (m, 2H), 2.66-2.54 (m, 2H), 2.38 (s, 3H), 1.54 (d, 6H); LCMS 500.9 (M+1)⁺.

### EXAMPLE 6

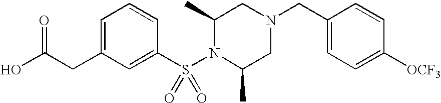

{3-[2,6-Dimethyl-4-(4-trifluoromethoxy-benzyl)-piperazine-1-sulfonyl]-phenyl}-acetic acid: The compound {3-[2,6-dimethyl-4-(4-trifluoromethoxy-benzyl)-piperazine-1-sulfonyl]-phenyl}-acetic acid was synthesized according to the procedure outlined in Example 1 using (3-chlorosulfonyl-phenyl)-acetic acid methyl ester. ¹H NMR (400 MHz, CD₃OD) δ 7.84 (s, 1H), 7.80-7.74 (m, 1H), 7.62 (d, 2H), 7.54-7.45 (m, 2H), 7.36 (d, 2H), 4.52-4.42 (m, 2H), 4.36 (s, 2H), 3.78 (s, 2H), 3.32-3.24 (m, 2H), 2.72-2.60 (m, 2H), 1.54 (d, 6H); LCMS 486.9 (M+1)⁺.

### EXAMPLE 7

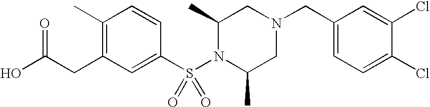

{5-[4-(3,4-Dichloro-benzyl)-2,6-dimethyl-piperazine-1-sulfonyl]-2-methyl-phenyl}-acetic acid: The compound {5-[4-(3,4-dichloro-benzyl)-2,6-dimethyl-piperazine-1-sulfonyl]-2-methyl-phenyl}-acetic acid was synthesized according to the procedure outlined in Example 1 using 3,4-dichlorobenzaldehyde. ¹H NMR (400 MHz, CD₃OD) δ 7.73-7.68 (m, 1H), 7.63 (dd, 1H), 7.58 (s, 1H), 7.55-7.48 (m, 1H), 7.40-7.28 (m, 2H), 4.28-4.18 (m, 2H), 3.86-3.72 (m, 4H), 2.92-2.80 (m, 2H), 2.39 (s, 3H), 2.28-2.16 (m, 2H), 1.46 (d, 6H); LCMS 487.8 (M+1)⁺.

### EXAMPLE 8

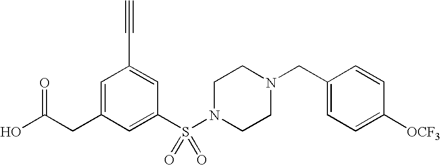

2-{3-Ethynyl-5-[-4-(4-trifluoromethoxybenzyl)piperazine-1-ylsulfonyl]phenyl}acetic acid:

### Step 1

3-Bromo-5-nitrobenzoic acid: Silver sulfate (11.7 g, 37.5 mmol) was added to a sulfuric acid solution (150 mL) of 3-nitrobenzoic acid (12.6 g, 75.4 mmol). The mixture was then treated with bromine (5.5 mL) and the solution stirred overnight at 130° C. After cooling the mixture to room temperature, the reaction was quenched with the addition of 300 mL of ice water. The mixture was filtered and washed with water (3×50 mL). The pH was adjusted to 10 by the addition of Na₂CO₃. Solids were removed by filtration and the pH of the filtrate was adjusted to two by the addition of HCl. The desired product was isolated by filtration and washed with water (3×50 mL) to afford 3-bromo-5-nitrobenzoic acid.

### Step 2

(3-Bromo-5-nitrophenyl)methanol: To sodium borohydride (1.1 g, 4.5 mmol) in THF (35 mL) was added the product of step 1 (3.5 g, 88.8 mmol) in several batches, while cooling to 0-5° C. Upon complete addition, a solution of boron trifluoride etherate (2.1 mL) in THF (10 mL) was added dropwise with stirring, while cooling to a temperature of 0° C. over 30 minutes. The resulting solution was stirred for 3 h at room temperature. The reaction was then quenched by the addition of 100 mL ice water. The resulting solution was extracted with EtOAc (3×100 mL) and the combined organic layers were washed with 10% Na₂CO₃. The mixture was dried over Na₂SO₄ and concentrated in vacuo to afford (3-bromo-5-nitrophenyl)methanol (3 g, 76%) as a white solid.

### Step 3

1-Bromo-3-(bromomethyl)-5-nitrobenzene: To a solution of the product of step 2 (3.0 g, 12.9 mmol) in CH₂Cl₂ (40 mL) was added tribromophosphine (15.5 mmol, 4.2 g) dropwise with stirring at 0° C. The resulting solution was stirred at room temperature. The mixture was then quenched by the addition ice water (200 mL). The resulting solution was extracted with CH₂Cl₂ and the combined organic layers were washed with saturated NaHCO₃. The mixture was dried over MgSO₄ and concentrated in vacuo. The residue was purified by chromatography on silica (20:1 EtOAc/PE) to afford 1-bromo-3-(bromomethyl)-5-nitrobenzene (2 g, 60%) as a yellow solid.

### Step 4

2-(3-Bromo-5-nitrophenyl)acetonitrile: To a solution of the product of step 3 (12.0 g, 40.7 mmol) in ethanol (180 mL) was added a solution of potassium cyanide (3.0 g, 46.2 mmol) in water (20 mL). The resulting solution was stirred overnight while the temperature was maintained at reflux in an oil bath. The mixture was concentrated in vacuo. To the residue was added water (200 mL). The resulting solution was extracted with CH₂Cl₂ (3×100 mL). The combined organic layers were dried over Na₂SO₄ and concentrated in vacuo to give 2-(3-bromo-5-nitrophenyl)acetonitrile (10 g, 31%) as a black oil.

### Step 5

2-(3-Bromo-5-nitrophenyl)acetic acid: To the product of step 4 (3.0 g, 12.5 mmol) was added sulfuric acid (7 mL), followed by acetic acid (7 mL) and water (7 mL). The resulting solution was heated at reflux overnight. The mixture was cooled and then quenched with the addition of water (50 mL). The resulting solution was extracted with EtOAc (3×30 mL) and the organic layers combined and concentrated in vacuo to afford 2-(3-bromo-5-nitrophenyl)acetic acid (2.0 g, 49%) as a brown solid.

### Step 6

Methyl 2-(3-bromo-5-nitrophenyl acetate: To a solution of the product of step 5 (2.0 g, 6.2 mmol) in MeOH (30 mL) was added sulfuric acid (1 mL). The resulting solution was heated at reflux overnight. The mixture was cooled and concentrated in vacuo. To the residue was added H₂O (20 mL). The resulting solution was extracted with EtOAc (2×20 mL) and the combined organic layers were dried over Na₂SO₄ and concentrated in vacuo to afford methyl 2-(3-bromo-5-nitrophenyl)acetate (2.8 g) as a black solid.

### Step 7

Methyl 2-(3-amino-5-bromophenyl)acetate: A mixture of the product of step 6 (1.0 g, 3.6 mmol) in water (35 mL) was heated to 70° C. To the mixture was added iron (1 g, 18.9 mmol, 2.8 g) followed by dropwise addition of acetic acid (1 g, 16.7 mmol) with stirring. The resulting solution was stirred for 1 h while the temperature was maintained at 95° C. in an oil bath. The resulting solution was cooled, filtered and extracted with EtOAc (3×30 mL). The combined organic layers were dried over Na₂SO₄ and concentrated in vacuo to afford methyl 2-(3-amino-5-bromophenyl)acetate (700 mg, 69%) as a black solid.

### Step 8

Methyl 2-(3-bromo-5-chlorosulfonyl-phenyl)acetate: To a solution of the product of step 7 (700 mg, 2.9 mmol) in acetonitrile (33 mL) at 0° C. was added concentrated hydrochloric acid (1.6 g) followed by dropwise addition of acetic acid (3.2 g) and a solution of sodium nitrite (240 mg) in water (5 mL). The solution was saturated with SO₂ and a solution of CuCl₂ (500 mg) in water (5 mL) was added, at 0° C. The resulting solution was stirred overnight at room temperature. The reaction mixture was quenched with the addition of 50 mL of H₂O/ice. The resulting solution was extracted with EtOAc (3×30 mL) and the combined organic layers were washed with water (3×100 mL), dried over Na₂SO₄, and concentrated in vacuo. The residue was purified by chromatography on silica (1:100 EtOAc/PE) to afford methyl 2-(3-bromo-5-chlorosulfonyl-phenyl)acetate (300 mg, 29%) as a white solid. ¹H NMR (400 MHz, CDCl₃) δ 8.05 (s, 1H), 7.86 (s, 1H), 7.79 (s, 1H), 3.70 (s, 2H), 3.72 (s, 3H).

### Step 9

Methyl 2-{3-bromo-5-[4-(4-trifluoromethoxybenzyl)piperazine-1-ylsulfonyl]phenyl}acetate: To a solution of 1-(4-trifluoromethoxybenzyl)piperazine (298 mg, 1.15 mmol) and triethylamine (0.40 mL, 2.86 mmol) in THF (3 mL) was added the product of step 8 (0.954 mmol, 0.313 g) as a solution in THF (3 mL). The mixture was stirred at room temperature for 1 h. The solvent was removed in vacuo and the crude material purified by chromatography on silica (0-30% EtOAc/hexanes) to afford methyl 2-{3-bromo-5-[4-(4-trifluoromethoxybenzyl)piperazine-1-ylsulfonyl]phenyl}acetate (228 mg).

### Step 10

Methyl 2-{3-[4-(4-trifluoromethoxybenzyl)piperazine-1-ylsulfonyl]-5-(trimethylsilylethynyl)phenyl}acetate: The product of step 9 (228 mg, 0.41 mmol), PdCl₂(PPh₃)₂ (29 mg, 0.041 mmol), PPh₃ (33 mg, 0.12 mmol), CuI (24 mg, 0.12 mmol) were combined in a microwave vial and N,N-dimethylformamide (2.5 mL), triethylamine (0.3 mL) were added followed by trimethylsilylacetylene (0.1 mL, 0.62 mmol). The mixture was heated in a microwave for 25 min at 120° C. The mixture was filtered through Celite and washed with EtOAc, extracted with saturated NH₄Cl, dried with Na₂SO₄, filtered and concentrated in vacuo. The crude material was purified by chromatography on silica (0-30% EtOAc/hexanes) to afford methyl 2-{3-[4-(4-trifluoromethoxybenzyl)piperazine-1-ylsulfonyl]-5-(trimethylsilylethynyl)phenyl}acetate (190 mg).

### Step 11

2-{3-Ethynyl-5-[-4-(4-trifluoromethoxybenzyl)piperazine-1-ylsulfonyl]phenyl}acetic acid: The product of step 10 was dissolved in 3 mL of MeOH/THF (2:3) to which 1.5 mL of 1 N LiOH was added The mixture was stirred for 30 minutes, then 1 N HCl was added and a precipitate formed. The reaction was extracted with EtOAc, dried and concentrated. The crude material was purified by reverse-phase HPLC to afford 2-{3-ethynyl-5-[-4-(4-trifluoromethoxybenzyl)piperazine-1-ylsulfonyl]phenyl}acetic acid. ¹H NMR (CD₃OD) δ 7.76 (s, 1H), 7.74 (s, 1H), 7.70 (s, 1H), 7.57 (d, 2H), 7.35 (s, 2H), 4.36 (s, 2H), 3.77 (s, 2H), 3.75 (s, 1H), 3.34 (m, 8H). MS (ESI): 483.

### EXAMPLE 9

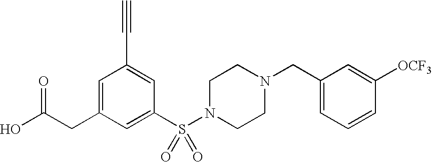

2-{3-Ethynyl-5-[4-(3-trifluoromethoxybenzyl)piperazine-1-ylsulfonyl]phenyl}acetic acid:

### Step 1

Methyl 2-{3-bromo-5-[4-(4-trifluoromethoxybenzyl)piperazine-1-ylsulfonyl]phenyl}acetate: To a solution of 1-(3-trifluoromethoxybenzyl)piperazine (210 mg, 0.81 mmol) and triethylamine (0.26 mL, 1.88 mmol) in THF (3 mL) was added (5-chlorosulfonyl-3-bromophenyl)-acetic acid methyl ester (220 mg, 0.67 mmol) (prepared in Example 9, step 8) as a solution in THF (2 mL). The mixture was stirred at room temperature for 1 h. The solvent was removed in vacuo and the crude material purified by chromatography on silica (0-30% EtOAc/hexanes) to afford methyl 2-{3-bromo-5-[4-(4-trifluoromethoxybenzyl)piperazine-1-ylsulfonyl]phenyl}acetate (289 mg).

### Step 2

Methyl 2-{3-[4-(3-trifluoromethoxybenzyl)piperazine-1-ylsulfonyl]-5-(trimethylsilylethynyl)phenyl}acetate: The product of step 1 (289 mg, 0.52 mmol), PdCl₂(PPh₃)₂ (37 mg, 0.052 mmol), PPh₃ (41 mg, 0.16 mmol), CuI (29 mg, 0.16 mmol) were combined in a microwave vial and N,N-dimethylformamide (2.5 mL), triethylamine (0.4 mL) were added followed by trimethylsilylacetylene (0.11 mL, 0.79 mmol). The mixture was heated in a microwave for 25 min at 120° C. The mixture was filtered through Celite and washed with EtOAc, extracted with saturated NH₄Cl, dried with Na₂SO₄, filtered and concentrated in vacuo. The crude material was purified by chromatography on silica (0-30% EtOAc/hexanes) to afford methyl 2-{3-[4-(3-trifluoromethoxybenzyl)piperazine-1-ylsulfonyl]-5-(trimethylsilylethynyl)phenyl}acetate (206 mg).

### Step 3

2-{3-Ethynyl-5-[4-(3-trifluoromethoxybenzyl)piperazine-1-ylsulfonyl]phenyl}acetic acid: The product of step 2 (206 mg, 0.36 mmol) was dissolved in 3 mL of MeOH/THF (2:3) to which 1.1 mL of 1 N LiOH was added The mixture was stirred for 30 min, then 1 N HCl was added and a precipitate formed. The mixture was extracted with EtOAc, dried and concentrated in vacuo. The crude material was purified by reverse-phase HPLC to afford the title compound. ¹H NMR (CD₃OD) δ 7.77 (s, 1H), 7.75 (s, 1H), 7.71 (s, 1H), 7.58 (t, 1H), 7.51 (d, 1H), 7.48 (s, 1H), 7.42 (d, 1H) 4.39 (s, 2H), 3.78 (s, 2H), 3.77 (s, 1H), 3.35 (m, 8H). MS (ESI): 483.

### EXAMPLE 10

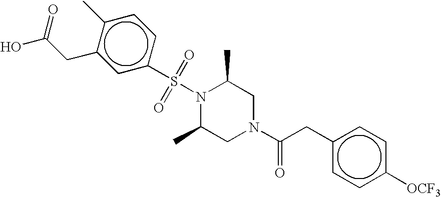

2-(5-((2R,6S)-2,6-dimethyl-4-(2-(4-(trifluoromethoxy)phenyl)acetyl)piperazin-1-ylsulfonyl)-2-methylphenyl)acetic acid: The compound, 2-(5-((2R,6S)-2,6-dimethyl-4-(2-(4-(trifluoromethoxy)phenyl)acetyl)piperazin-1-ylsulfonyl)-2-methylphenyl)acetic acid, was synthesized according to the procedure outlined in Example 1, using 1-(cis-3,5-dimethylpiperazin-1-yl)-2-(4-(trifluoromethoxy)phenyl)ethanone. ¹H NMR (400 MHz, CD₃OD) δ 7.71 (d, 1H), 7.65 (dd, 1H), 7.38 (d, 1H), 7.32 (d, 2H), 7.21 (d, 2H), 4.19-4.08 (m, 3H), 3.74-3.70 (m, 5H), 3.02 (dd, 1H), 2.59 (dd, 1H), 2.38 (s, 3H), 1.26 (d, 3H), 1.17 (d, 3H). LCMS: 529.8 (M+1)⁺.

The following compounds can be made using the methods as described above and when made should have activity similar to those made above.

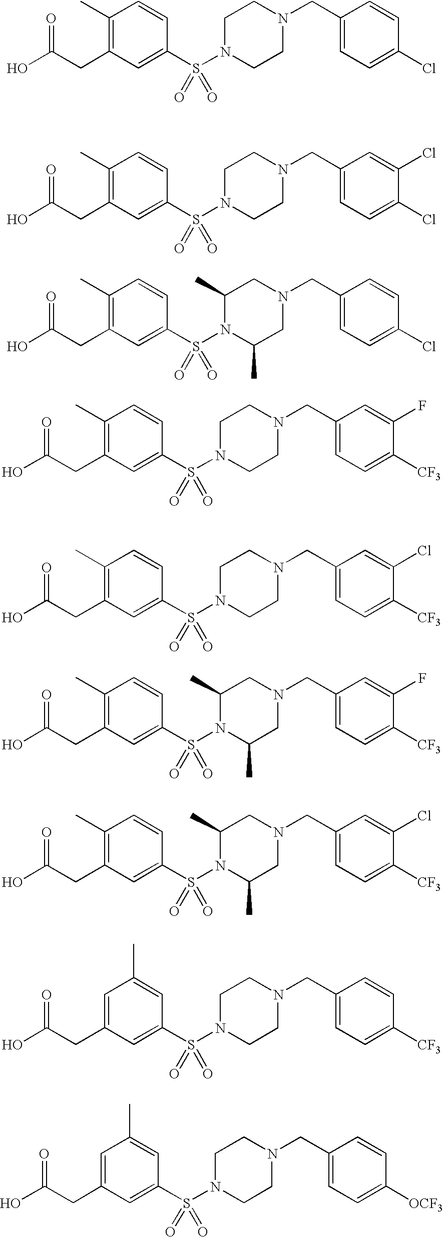

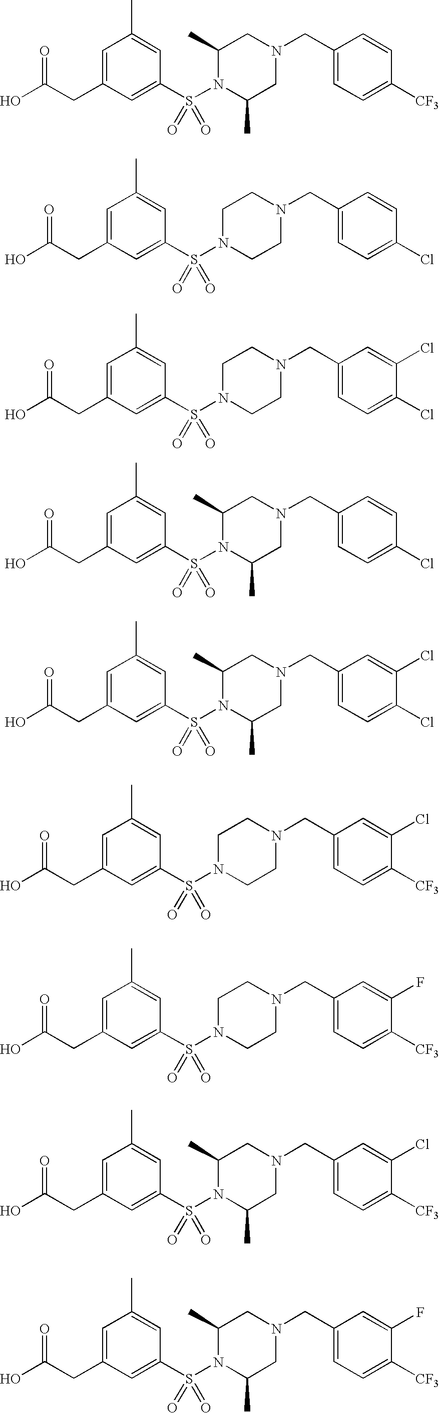

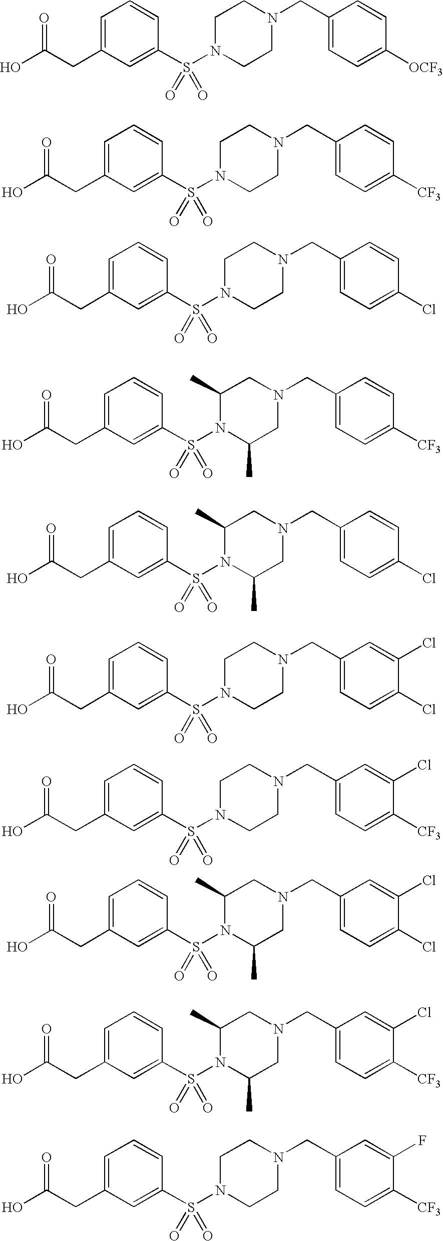

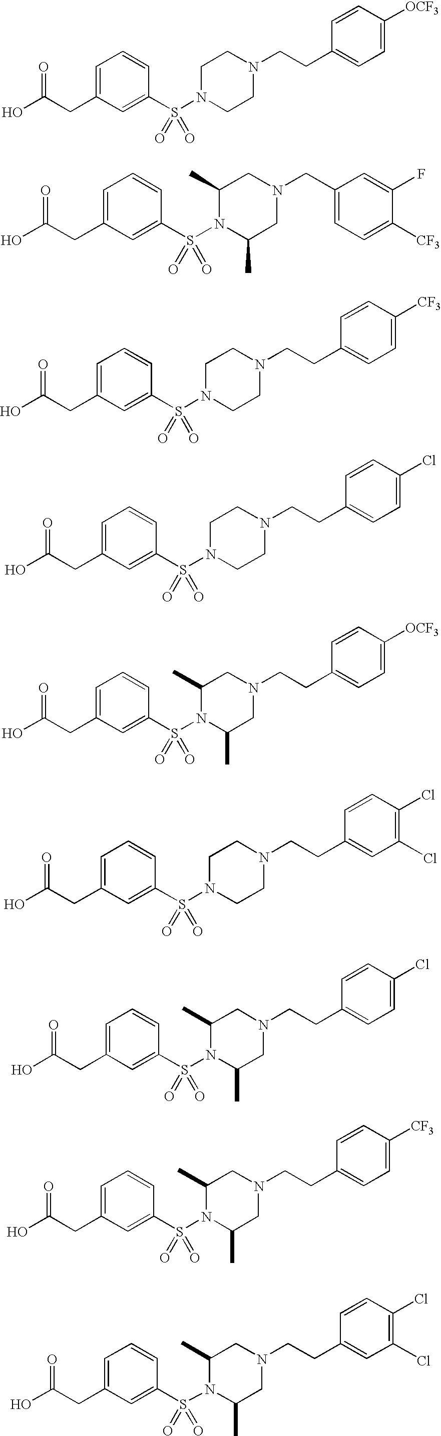

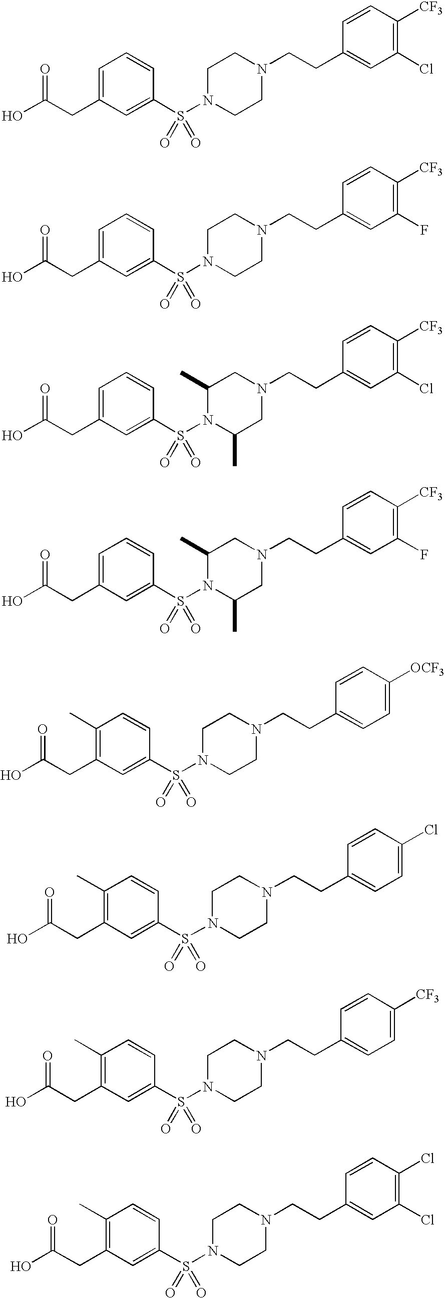

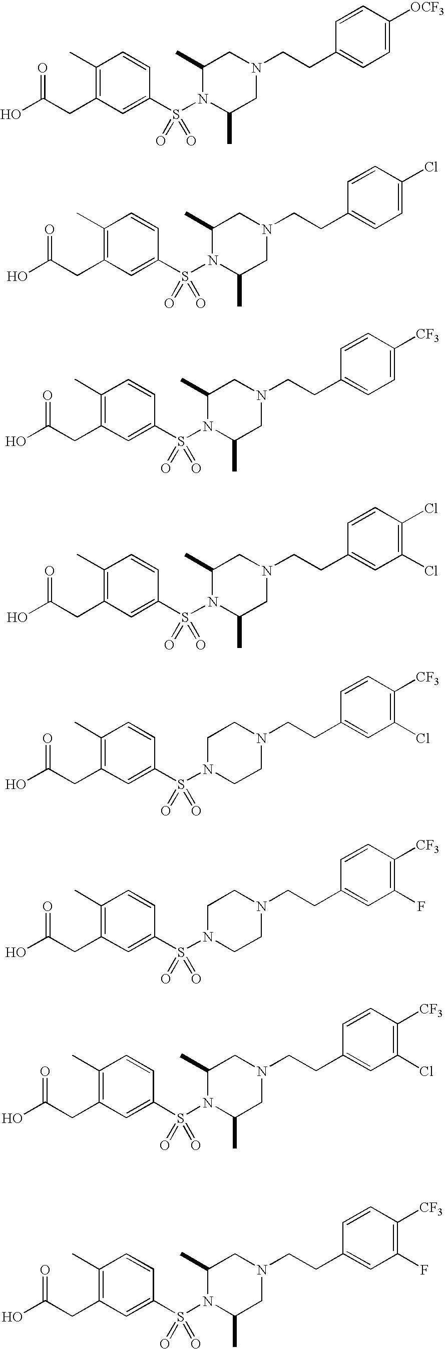

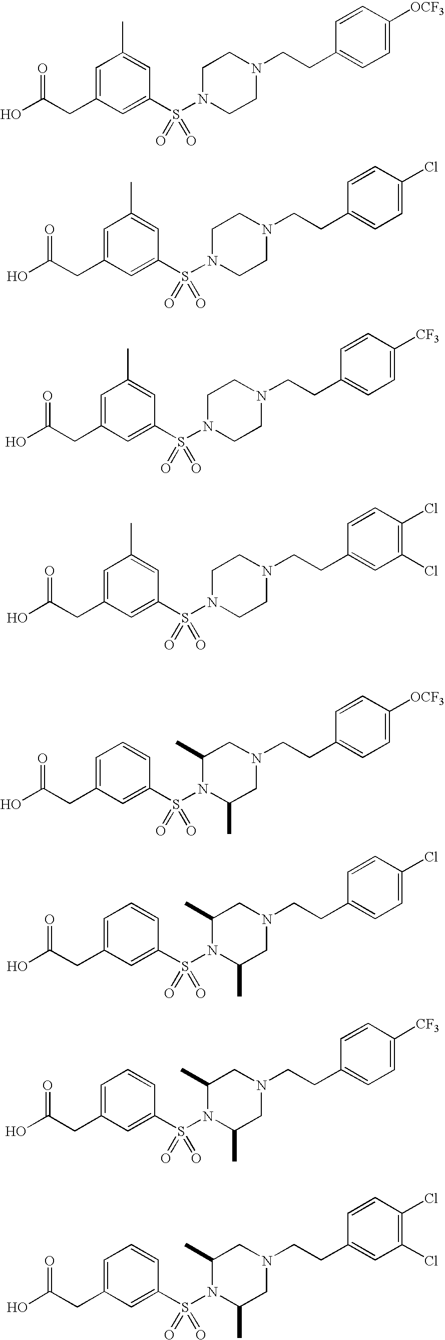

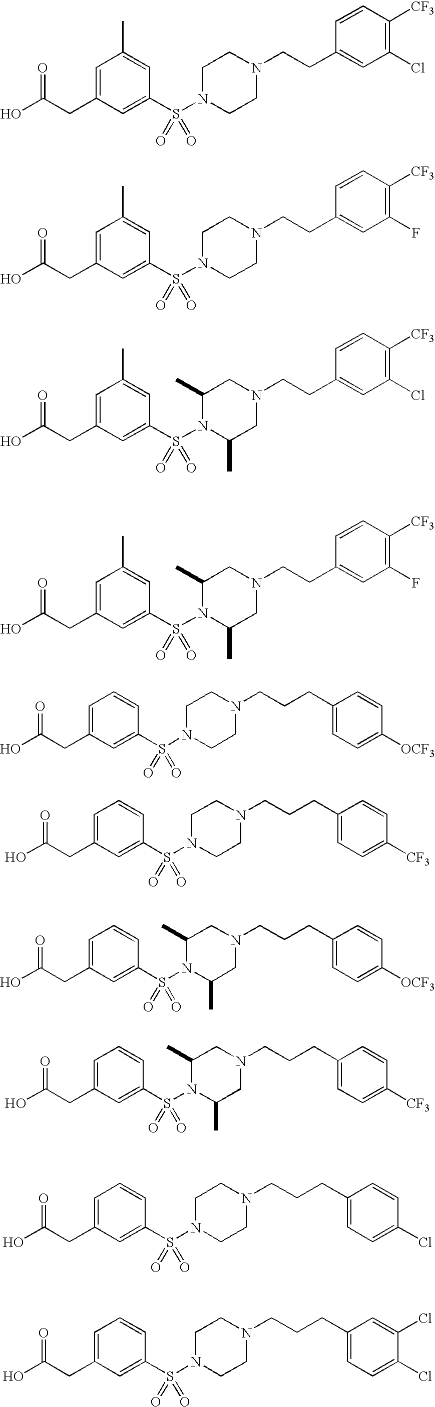

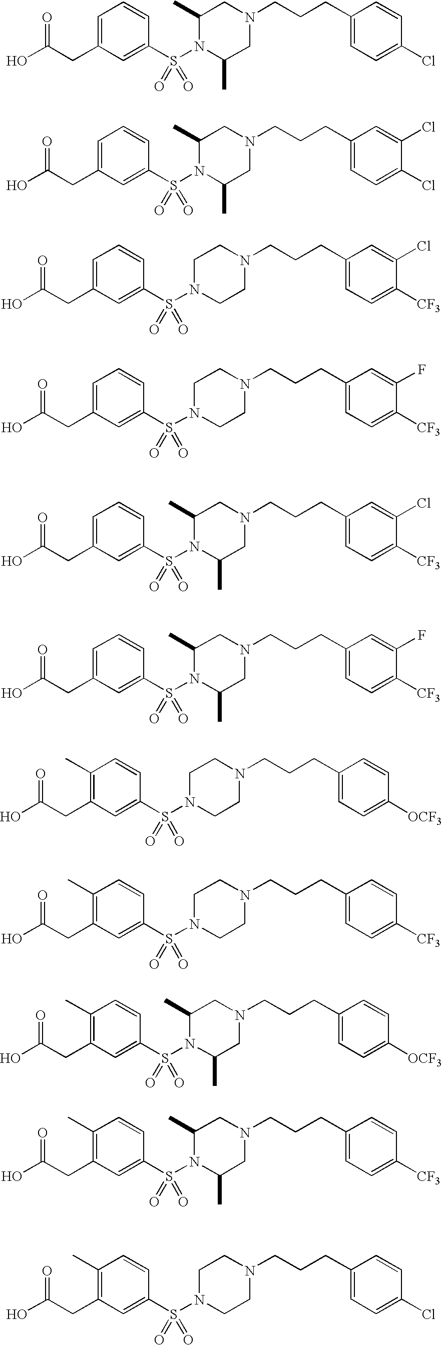

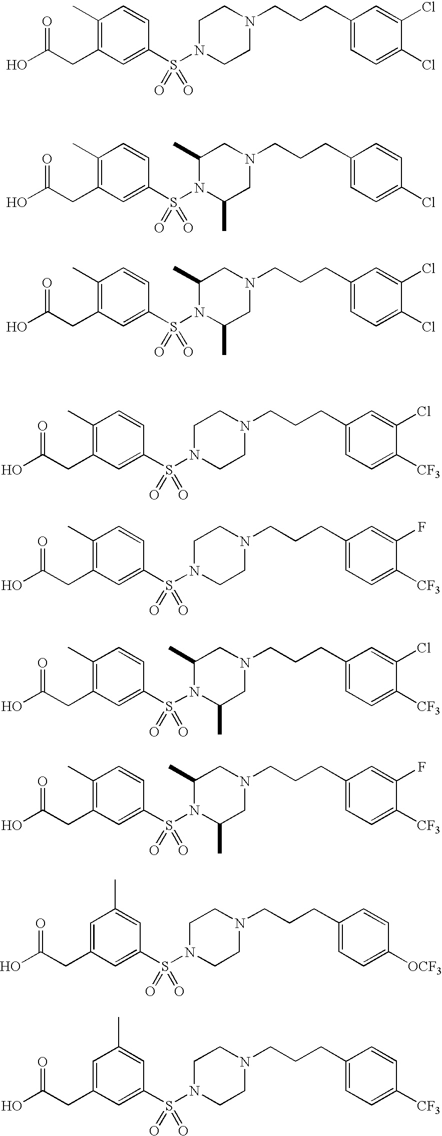

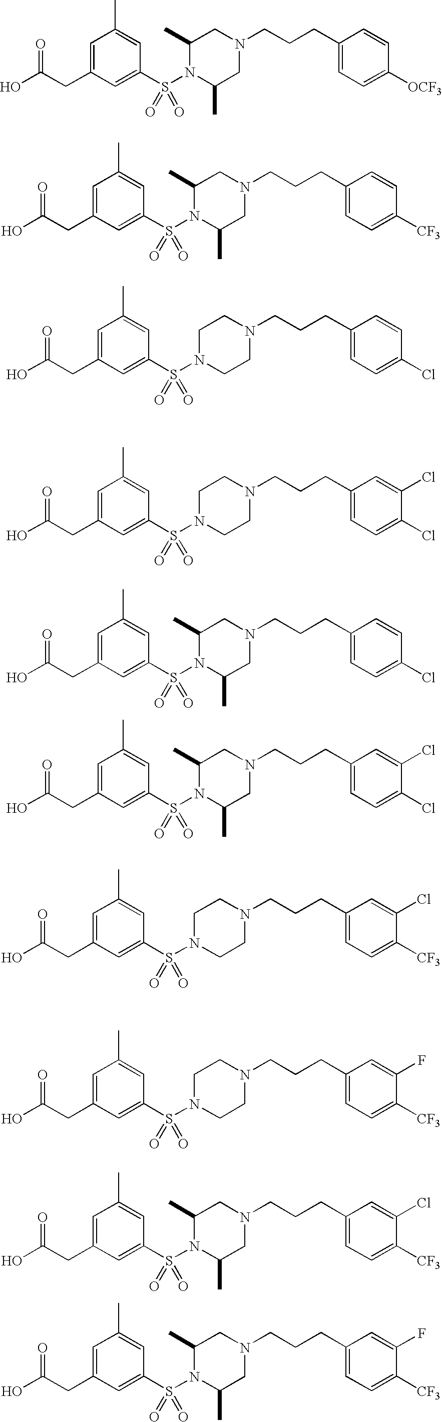

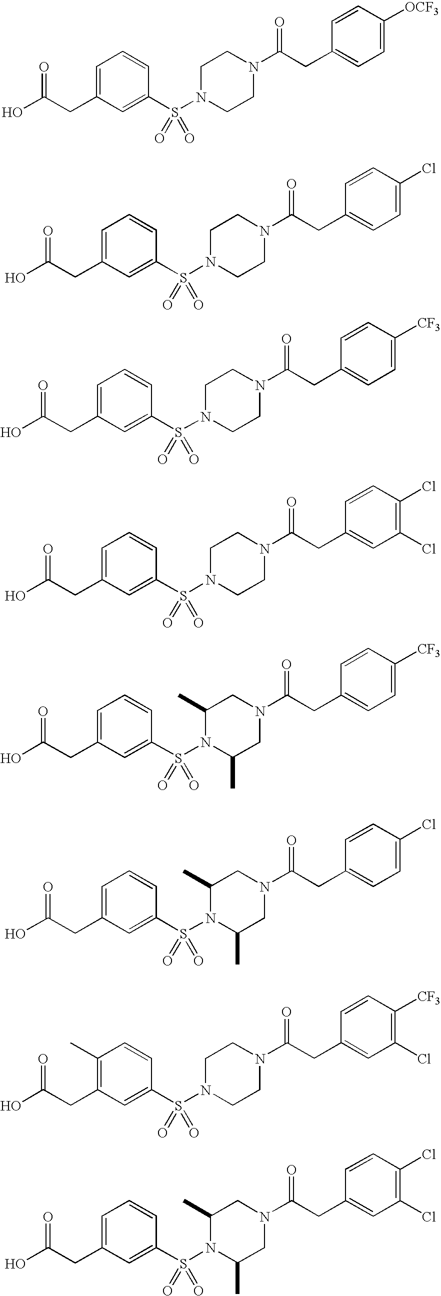

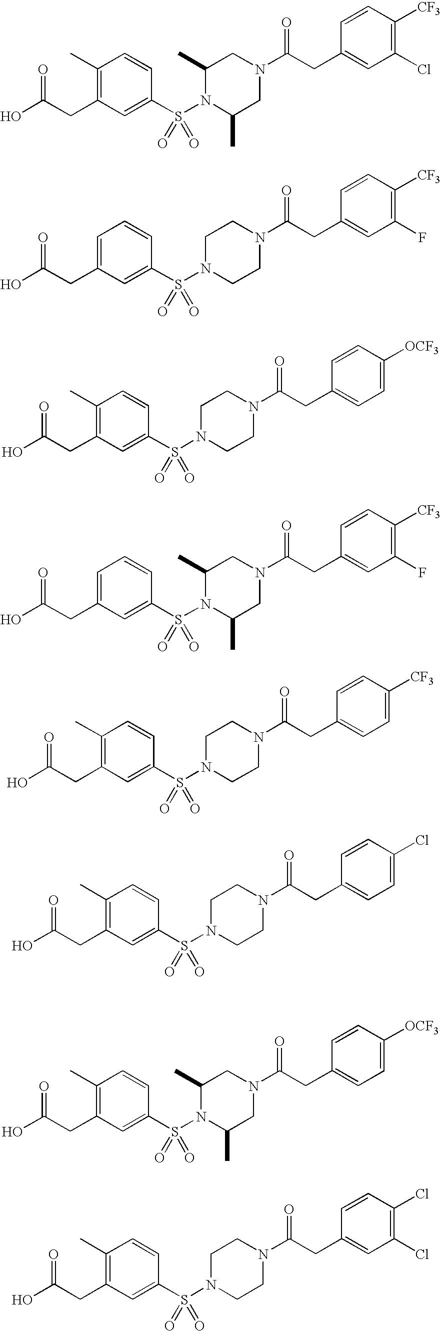

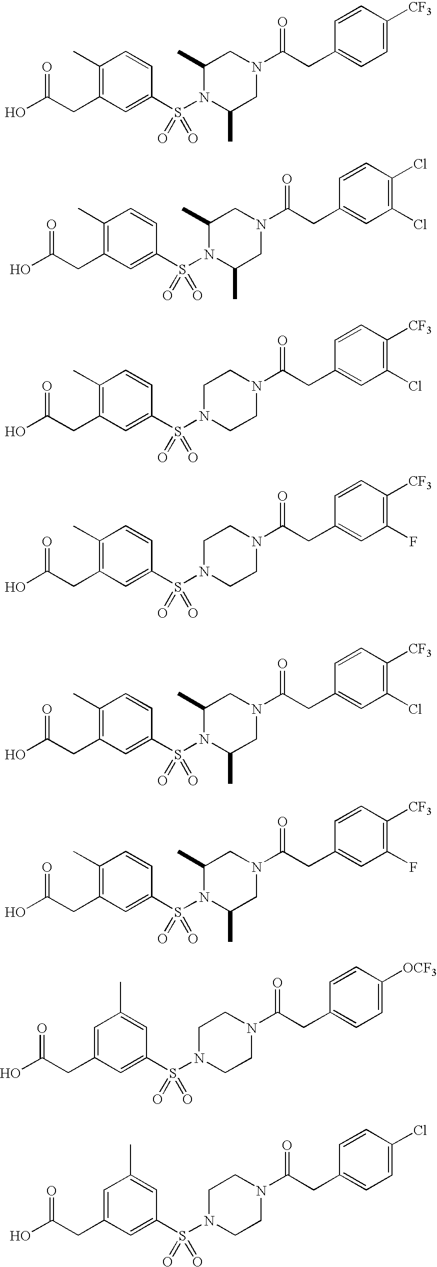

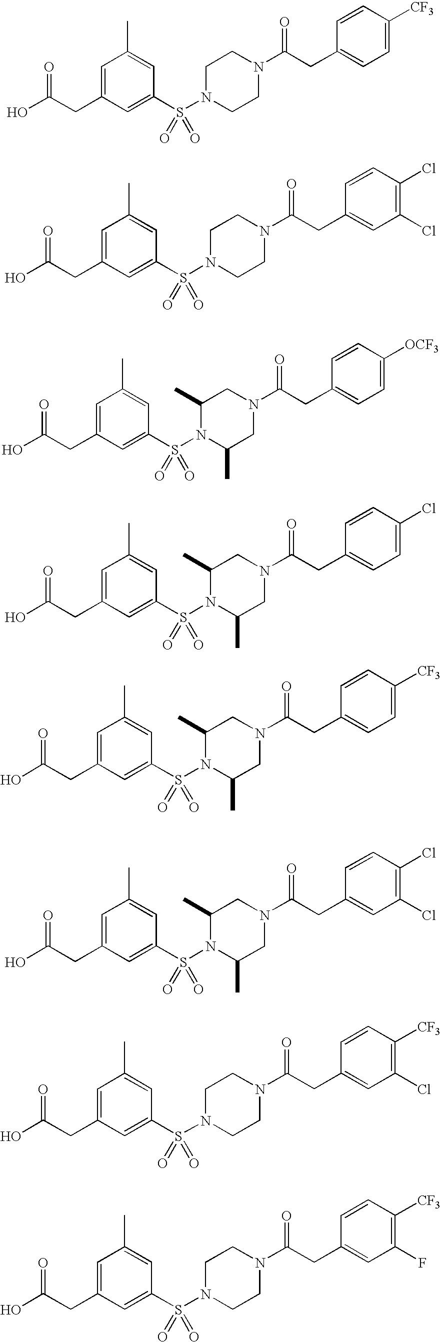

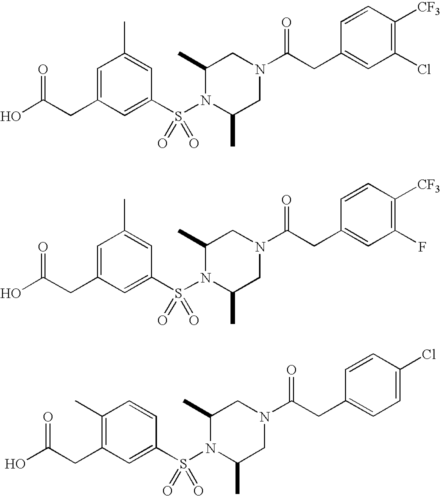

The compounds in examples 1-10 have been shown to be PPAR modulators by the following assay.

### Biological Activity

Compounds may be screened for functional potency in transient transfection assays in CV-1 cells for their ability to activate the PPAR subtypes (transactivation assay). A previously established chimeric receptor system was utilized to allow comparison of the relative transcriptional activity of the receptor subtypes on the same synthetic response element and to prevent endogenous receptor activation from complicating the interpretation of results. See, for example, Lehmann, J. M.; Moore, L. B.; Smith-Oliver, T. A; Wilkinson, W. O.; Willson, T. M.; Kliewer, S. A., An antidiabetic thiazolidinedione is a high affinity ligand for peroxisome proliferator-activated receptor δ (PPARδ), J. Biol. Chem., 1995, 270, 12953-6. The ligand binding domains for murine and human PPAR-alpha, PPAR-gamma, and PPAR-delta are each fused to the yeast transcription factor GAL4 DNA binding domain. CV-1 cells were transiently transfected with expression vectors for the respective PPAR chimera along with a reporter construct containing four or five copies of the GAL4 DNA binding site driving expression of luciferase. After 8-16 h, the cells are replated into multi-well assay plates and the media is exchanged to phenol-red free DME medium supplemented with 5% delipidated calf serum. 4 hours after replating, cells were treated with either compounds or 1% DMSO for 20-24 hours. Luciferase activity was then assayed with Britelite (Perkin Elmer) following the manufacturer's protocol and measured with either the Perl in Elmer Viewlux or Molecular Devices Acquest (see, for example, Kliewer, S. A., et. al. Cell 1995, 83, 813-819). Rosiglitazone is used as a positive control in the PPARδ assay. Wy-14643 and GW7647 is used as a positive control in the PPARδ assay. GW501516 is used as the positive control in the PPARδ assay.

Compounds of Examples 1-10 were assayed to measure their biological activity with respect to their EC₅₀ values and efficacy for modulating PPAR-alpha, PPAR-gamma, and PPAR-delta as set forth in Table 1.

TABLE 1
Biological Activity

| | PPAR alpha | PPAR delta | PPAR gamma |
|---|---|---|---|
| | A = >100 μM | A = >100 μM | A = >100 μM |
| | B = 5-100 μM | B = 5-100 μM | B = 5-100 μM |
| Example | C = <5 μM | C = <5 μM | C = <5 μM |
| 1 | C | C | A |
| 2 | C | C | B |
| 3 | C | C | C |
| 4 | C | C | C |
| 5 | B | C | A |
| 6 | B | C | C |
| 7 | C | C | A |
| 8 | C | C | C |
| 9 | C | C | C |
| 10 | A | C | A |

From the foregoing description, one skilled in the art can easily ascertain the essential characteristics of this invention, and without departing from the spirit and scope thereof, can make various changes and modifications of the invention to adapt it to various usages and conditions.

We claim:

1. A compound of structural Formula (I) 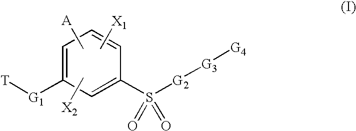
or salt, ester, or prodrug thereof, wherein:
T is —C(O)OH, —C(O)NH₂, or tetrazole;
G₁ is selected from the group consisting of —(CR₁R₂)—, -Z(CR₁R₂)—, —(CR₁R₂)ₙZ, and —(CR₁R₂)ᵣZ(CR₁R₂)ₛ—;
Z is O, S, or NR₃;
n is 1 to 5;
r and s are each independently 0 or 1;
R₁ and R₂ are each independently selected from the group consisting of hydrogen, halogen, optionally substituted lower alkyl, optionally substituted lower heteroalkyl, optionally substituted lower alkoxy, or together may form an optionally substituted cycloalkyl;
R₃ is selected from the group consisting of hydrogen, optionally substituted lower alkyl, and optionally substituted heteroalkyl;
A, X₁, and X₂ are each independently selected from the group consisting of hydrogen, optionally substituted lower alkyl, optionally substituted cycloalkyl, halogen, optionally substituted heteroalkyl, optionally substituted cycloheteroalkyl, optionally substituted lower alkynyl, perhaloalkyl, perhaloalkoxy, hydroxy, optionally substituted lower alkoxy, nitro, cyano, and optionally substituted amino;
G₂ is a 5, 6, or 7-membered carbocycle or heterocycle having the structure 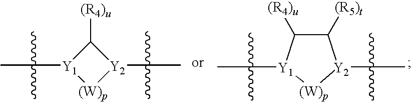
Y₁ and Y₂ are each independently selected from the group consisting of CR₆ and N;
W is selected from the group consisting of —CR₇R₈—, and —CR₇— joined together with Y₁ or Y₂ by a double bond;
p is 1, 2 or 3;
R₄ and R₅ are each independently selected from the group consisting of hydrogen, optionally substituted lower alkyl, halogen, lower perhaloalkyl, hydroxy, optionally substituted heteroalkyl, optionally substituted cycloalkyl, optionally substituted lower alkoxy, nitro, cyano, lower perhaloalkoxy, NH₂, and —C(O)—O—R₁₁; or, when both Y₁ and Y₂ are N, one of W may be taken together with one of R₄ or R₅ to form an optionally substituted 1- or 2-carbon bridge;
R₁₁ is selected from the group consisting of hydrogen and optionally substituted lower alkyl;
R₆ is selected from the group consisting of hydrogen, optionally substituted lower alkyl, hydroxy, and lower perhaloalkyl, or is null when Y₁ or Y₂ is joined to W by a double bond;
u and t are each independently 1 or 2;
R₇ and R₈ are each independently selected from the group consisting of hydrogen, optionally substituted lower alkyl, optionally substituted cycloalkyl, optionally substituted heteroalkyl, hydroxy, optionally substituted lower alkoxy, cyano, halogen, lower perhaloalkyl, NH₂, and a moiety which taken together with R₄ and R₅ forms a 1 or 2 carbon bridge;
G₃ is selected from the group consisting of —(CR₉R₁₀)ₘ—, and —(CR₉R₁₀)_{q}J(CR₉R₁₀)ₖ—;
J is O, S, SO₂, C(O) or NR₁₂;
m is 1 to 3;
q is 0 to 3;
k is 0 to 3;
R₉ and R₁₀ are each independently selected from the group consisting of hydrogen, optionally substituted lower alkyl, optionally substituted lower alkoxy, lower perhaloalkyl, cyano, and nitro;
R₁₂ is selected from the group consisting of hydrogen, optionally substituted lower alkyl, and optionally substituted heteroalkyl;
G₄ is selected from the group consisting of hydrogen, optionally substituted aryl, optionally substituted heteroaryl, optionally substituted cycloalkyl, optionally substituted cycloheteroalkyl, optionally substituted cycloalkenyl, optionally substituted fused aryl, optionally substituted fused heteroaryl, and optionally substituted fused cycloalkyl; and
with the provisos that:
r and s are not both 0;
R₄ is not hydroxy or NH₂ when Y₁ is N;
R₅ is not hydroxy or NH₂ when Y₂ is N;
R₇ and R₈ are not hydroxy or NH₂ when attached to a ring carbon atom adjacent to a ring nitrogen atom;
when G₃ is —(CR₉R₁₀)ₘ—, then none of R₉, R₁₀ and G₄ are selected from the group consisting of unsubstituted phenyl, 4-fluorophenyl, and cyclohexyl;
when G₃ is —(CR₉R₁₀)_{q}J(CR₉R₁₀)ₖ, J is C(O), and q and k are both 0, G₄ is not 2-furanyl; and
when G₄ is said optionally substituted cycloheteroalkyl, said optional substituents are non-cyclic.

2. The compound as recited in claim 1, wherein:
G₁ is —(CR₁R₂)ₙ—; and
R₁ and R₂ are each independently selected from the group consisting of hydrogen, halogen, optionally substituted lower alkyl, optionally substituted lower heteroalkyl, and optionally substituted lower alkoxy.

3. The compound as recited in claim 2, wherein R₁ and R₂ are each independently selected from the group consisting of hydrogen, methyl, ethyl, and propyl.

4. The compound as recited in claim 3, wherein R₁ and R₂ are hydrogen.

5. The compound of claim 4 wherein T is —C(O)OH.

6. The compound as recited in claim 5 wherein n=1.

7. The compound as recited in claim 6 wherein A, X₁, and X₂ are each independently selected from the group consisting of hydrogen, optionally substituted lower alkyl, lower perhaloalkyl, and halogen.

8. The compound as recited in claim 7 having a structural formula selected from the group consisting of:

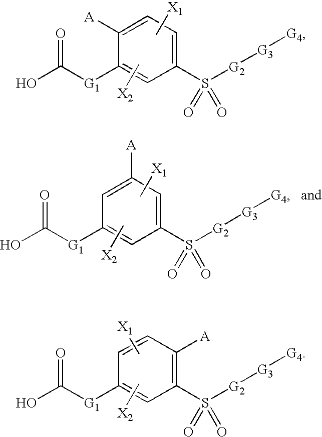

9. The compound as recited in claim 8, wherein A is either hydrogen or methyl and X₁ and X₂ are hydrogen.

10. The compound as recited in claim 9 wherein G₃ is —(CR₉R₁₀)ₘ—.

11. The compound as recited in claim 10 wherein R₉ and R₁₀ are hydrogen.

12. The compound as recited in claim 11 wherein m is 1 to 2.

13. The compound as recited in claim 9 wherein G₃ is —(CR₉R₁₀)_{q}J(CR₉R₁₀)ₖ—.

14. The compound as recited in claim 13 wherein R₉ and R₁₀ are hydrogen.

15. The compound as recited in claim 14 wherein q is 0.

16. The compound as recited in claim 15 wherein J is C(O).

17. The compound as recited in claim 9 wherein:
G₄ has the structure: 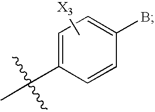
B is selected from the group consisting of hydrogen, halogen, optionally substituted lower alkyl, optionally substituted heteroalkyl, optionally substituted cycloalkyl, optionally substituted heteroalkyl, optionally substituted cycloheteroalkyl, optionally substituted lower alkynyl, optionally substituted lower alkoxy, lower perhaloalkyl, lower perhaloalkoxy, nitro, cyano, hydroxy, NH₂ and —CO₂R₁₁;
R₁₁ is selected from the group consisting of optionally substituted lower alkyl and hydrogen; and
X₃ is selected from the group consisting of hydrogen, halogen, optionally substituted lower alkyl, optionally substituted heteroalkyl, optionally substituted cycloalkyl, optionally substituted cycloheteroalkyl, optionally substituted lower alkynyl, optionally substituted lower alkoxy, lower perhaloalkyl, lower perhaloalkoxy, nitro, cyano, hydroxy, NH₂ and —CO₂R₁₁.

18. The compound as recited in claim 17 wherein B is selected from the group consisting of hydrogen, halogen, lower perhaloalkyl and lower perhaloalkoxy.

19. The compound as recited in claim 18 wherein G₃ is —(CR₉R₁₀)ₘ—.

20. The compound as recited in claim 19 wherein R₉ and R₁₀ are hydrogen.

21. The compound as recited in claim 20 wherein m is 1.

22. The compound as recited in claim 8, wherein:
G₂ has the structure 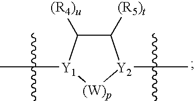
Y₁ and Y₂ are both N;
each W is —CR₇R₈—;
p is 2; and
with the proviso that at least one of R₄, R₅, R₇, and R₈ is not hydrogen.

23. The compound as recited in claim 22, wherein said at least one of R₄, R₅, R₇, and R₈ is lower alkyl.

24. The compound as recited in claim 23, wherein said at least one of R₄, R₅, R₇, and R₈ is methyl.

25. The compound as recited in claim 24, wherein at least two of R₄, R₅, R₇, and R₈ are methyl.

26. The compound as recited in claim 25, wherein R₄ and R₇ are methyl and are attached to the piperazine ring at the 2 and 6 positions.

27. The compound as recited in claim 26, wherein the R₄ and R₇ methyl groups are oriented cis to each other.

28. The compound as recited in claim 25 wherein
G₃ is —(CR₉R₁₀)ₘ—;
R₉ and R₁₀ are hydrogen; and
m is 1 to 2.

29. The compound as recited in claim 25 wherein
G₃ is —(CR₉R₁₀)_{q}J(CR₉R₁₀)ₖ—;
R₉ and R₁₀ are hydrogen;
q is 0; and
J is C(O).

30. A compound of structural Formula (II) 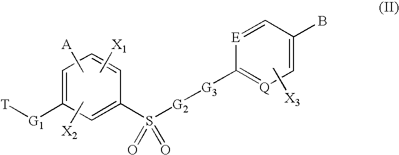
or salt, ester, or prodrug thereof, wherein:
T is —C(O)OH, —C(O)NH₂, or tetrazole;
G₁ is —(CR₁R₂)ₙ—;
n is 1 to 5;
R₁ and R₂ are each independently selected from the group consisting of hydrogen, halogen, optionally substituted lower alkyl, optionally substituted lower heteroalkyl, and optionally substituted lower alkoxy;
A, X₁, and X₂ are each independently selected from the group consisting of hydrogen, optionally substituted lower alkyl, optionally substituted cycloalkyl, halogen, optionally substituted heteroalkyl, optionally substituted cycloheteroalkyl, optionally substituted lower alkynyl, perhaloalkyl, perhaloalkoxy, hydroxy, optionally substituted lower alkoxy, nitro, cyano, and optionally substituted amino;
G₂ is a 5, 6, or 7-membered carbocycle or heterocycle having the structure 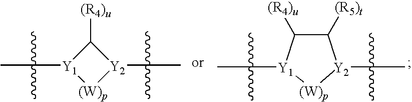
Y₁ and Y₂ are each independently selected from the group consisting of CR₆ and N;
W is selected from the group consisting of —CR₇R₉—, and —CR₇— joined together with Y₁ or Y₂ by a double bond;
p is 1, 2 or 3;
R₄ and R₅ are each independently selected from the group consisting of hydrogen, optionally substituted lower alkyl, halogen, lower perhaloalkyl, hydroxy, optionally substituted heteroalkyl, optionally substituted cycloalkyl, optionally substituted lower alkoxy, nitro, cyano, lower perhaloalkoxy, NH₂, and —C(O)—O—R₁₁; or, when both Y₁ and Y₂ are N, one of W may be taken together with one of R₄ or R₅ to form an optionally substituted 1- or 2-carbon bridge;
R₁₁ is selected from the group consisting of hydrogen and optionally substituted lower alkyl;
R₆ is selected from the group consisting of hydrogen, optionally substituted lower alkyl, hydroxy, and lower perhaloalkyl, or is null when Y₁ or Y₂ is joined to W by a double bond;
u and t are each independently 1 or 2;
R₇ and R₈ are each independently selected from the group consisting of hydrogen, optionally substituted lower alkyl, optionally substituted cycloalkyl, optionally substituted heteroalkyl, hydroxy, optionally substituted lower alkoxy, cyano, halogen, lower perhaloalkyl, NH₂, and a moiety which taken together with R₄ and R₅ forms a 1 or 2 carbon bridge;
G₃ is selected from the group consisting of —(CR₉R₁₀)ₘ—, and —(CR₉R₁₀)_{q}J(CR₉R₁₀)ₖ—;
J is O, S, SO₂, C(O) or NR₁₂;
m is 1 to 3;
q is 0 to 3;
k is 0 to 3;
R₉ and R₁₀ are each independently selected from the group consisting of hydrogen, optionally substituted lower alkyl, optionally substituted lower alkoxy, lower perhaloalkyl, cyano, and nitro;
R₁₂ is selected from the group consisting of hydrogen, optionally substituted lower alkyl, and optionally substituted heteroalkyl;
E and Q are each independently selected from the group consisting of CR₁₃ and N;
each R₁₃ is selected from the group consisting of hydrogen, halogen, optionally substituted lower alkyl, optionally substituted heteroalkyl, optionally substituted cycloalkyl, optionally substituted cycloheteroalkyl, optionally substituted lower alkynyl, optionally substituted lower alkoxy, lower perhaloalkyl, lower perhaloalkoxy, nitro, cyano, hydroxy, NH₂ and —CO₂R₁₁;
X₃ is selected from the group consisting of hydrogen, halogen, optionally substituted lower alkyl, optionally substituted heteroalkyl, optionally substituted cycloalkyl, optionally substituted cycloheteroalkyl, optionally substituted lower alkynyl, optionally substituted lower alkoxy, lower perhaloalkyl, lower perhaloalkoxy, nitro, cyano, hydroxy, NH₂ and —CO₂R₁₁; and
B is selected from the group consisting of optionally substituted lower alkyl, optionally substituted heteroalkyl, optionally substituted cycloalkyl, optionally substituted heteroalkyl, optionally substituted cycloheteroalkyl, optionally substituted lower alkynyl, optionally substituted lower alkoxy, lower perhaloalkyl, lower perhaloalkoxy, nitro, cyano, hydroxy, chloro, bromo, NH₂ and —CO₂R₁₁.

31. The compound as recited in claim 1, selected from the group consisting of Examples 1 to 10.

32. A compound or composition as recited in claim 1 for use as a medicament.

33. A compound or composition as recited in claim 1 for use in the manufacture of a medicament for the prevention or treatment of a disease or condition ameliorated by the modulation of PPAR.

34. The compound as recited in claim 33, wherein said PPAR is PPARδ.

35. A pharmaceutical composition comprising a compound as recited in claim 1 together with a pharmaceutically acceptable carrier.

36. A method of modulation of PPAR comprising contacting PPAR with a compound as recited in claim 1.

37. The method as recited in claim 36, wherein said PPAR is PPARδ.

38. A method of treatment of a PPAR-mediated disease comprising the administration of a therapeutically effective amount of a compound as recited in claim 1 to a patient in need thereof.

39. The method as recited in claim 38 wherein said PPAR is PPARδ.

40. The method as recited in claim 39 wherein said disease is selected from the group consisting of obesity, diabetes, metabolic syndrome, and hyperlipidemia.

41. A method of treatment of a PPAR-mediated disease comprising the administration of:
a. a therapeutically effective amount of a compound as recited in claim 1; and
b. another therapeutic agent.

42. The method as recited in claim 41 wherein said other agent is selected from the group consisting of rosiglitazone, pioglitazone, ezetimibe, or a statin.

43. A method for achieving an effect in a patient comprising the administration of a therapeutically effective amount of a compound as recited in claim 1 to a patient, wherein the effect is selected from the group consisting of upregulation of expression of GLUT4 in adipose tissue, reduction of expression of NPC1L1, raising of HDL, lowering of LDLc, shifting of LDL particle size from small dense to normal LDL, inhibition of cholesterol absorption, reduction of triglycerides, decrease of insulin resistance, lowering of blood pressure, promotion of wound healing, reduction of scarring, and treatment of a PPARδ-mediated disease.

44. The method as recited in claim 43 wherein said PPARδ-mediated disease is selected from the group consisting of obesity, diabetes, hyperinsulinemia, metabolic syndrome X, dyslipidemia, hypercholesterolemia, cardiovascular disease, vascular disease, atherosclerosis, coronary heart disease, cerebrovascular disease, heart failure, peripheral vessel disease, hyperproliferative disorders, cancers, inflammatory diseases, asthma, rheumatoid arthritis, osteoarthritis, disorders associated with oxidative stress, inflammatory response to tissue injury, psoriasis, ulcerative colitis, dermatitis, autoimmune disease, opthalmologic diseases, dry eye, macular degeneration, closed angle glaucoma, wide angle glaucoma, inflammation of the eye, and pain of the eye.
